(19) United States
(12) Patent Application Publication
(10) Pub. No.: US 2009/0181889 A1
(43) Pub. Date: Jul. 16, 2009

(54) COMPOSITIONS AND METHODS FOR DETECTION AND TREATMENT OF HUMAN HERPESVIRUS (HHV)-6

(75) Inventors: Stephen Dewhurst, Rochester, NY (US); Zhu Zhen, Bellevue, WA (US); Birgit Bradel-Tretheway, Rochester, NY (US)

Correspondence Address: FISH & RICHARDSON P.C., P.O BOX 1022, Minneapolis, MN, 55440-1022, US

(73) Assignee: UNIVERSITY OF ROCHESTER, Rochester, NY (US)

(21) Appl. No.: 12/281,849

(22) PCT Filed: Mar. 7, 2007
(86) PCT No.: PCT/US07/63514

§ 371 Date: Dec. 12, 2008

Related U.S. Application Data
(60) Provisional application No. 60/780,486, filed on Mar. 8, 2006.

(51) Int. Cl. A61K 38/16; A61K 31/7088; A61K 38/02; A61K 31/727; G01N 33/567; C07K 14/00; A61P 31/12

(52) U.S. Cl. 514/12; 514/44; 514/2; 514/56; 435/7.21; 530/300

(57) ABSTRACT

Disclosed herein are compositions and methods for detection and treatment of human herpesvirus (HHV)-6.

### CROSS-REFERENCE TO RELATED APPLICATIONS

This application claims the benefit of U.S. Provisional Application No. 60/780,486, filed Mar. 8, 2006.

### STATEMENT REGARDING FEDERALLY SPONSORED RESEARCH

This invention was made with government support under Grant RO1 DE14194 awarded by the National Institutes of Health (NIH). The government has certain rights in the invention.

### BACKGROUND OF THE INVENTION

Human herpesvirus 6 (HHV-6) was first isolated in 1986 from patients with lymphoproliferative disorders (Salahuddin, et al. 1986) and later was identified as the causative agent of roseola infantum (Yamanishi, et al. 1988) and of acute febrile illness (Pruksananonda, et al. 1992; Zerr, et al. 2005) in young children. Following primary infection, the virus is able to establish a highly successful state of coexistence with the host, resulting in persistent infection with occasional but generally nonsymptomatic reactivation (Caserta, et al. 2004; Hail et al. 1994). However, the virus can cause rare, serious complications in immunocompromised hosts or in the context of stem cell transplantation, including encephalitis, hepatitis, and bone marrow suppression (Clark, et al. 2003; Wang, et al. 1999; Zerr, et al. 2001). There are two variants of this virus, 6A and 6B, which have characteristic differences in their cell tropism mid biological properties (Ablashi, et al. 1991; Aubin, et al. 1991; Dewhurst, et al. 1992; Schirmer, et al. 1991) as well as approximately 10% overall sequence divergence at the genomic level (Dominguez, et al. 1999; Compels, et al. 1995; Isegawa, et al. 1999).

Two genetically and phenotypically distinct variants of HHV-6 have been reported: HHV-6A and HHV-6B. The two variants possess distinct biologic properties and cellular tropism (Ablashi, et al. 1993; Ablashi, et al. 1991; Dewhurst, et al. 1992; Schirmer, et al. 1991), as well as probable differences in their pathogenic properties, tissue distribution and epidemiology (Carrigan, et al. 1996; Cone, et al. 1996; Dewhurst, et al. 1993; Hall, et al. 1998; Kasolo, et al. 1997; Razonable, et al 2002; Soldan, et al. 2000).

In vitro, only HHV-6A infects and depletes CD8+ T lymphocytes in cultured human tonsil tissue fragments (Grivel, et al. 2003) and only HHV-6A productively infects cultured primary human progenitor-derived astrocytes (Donati, et al. 2005). There are also variant-specific differences in the cytopathic effects of HHV-6 on cultured, CNS-derived cells (De Bolle, et al. 2005; Kong, et al. 2003), as well as differences in the propensity of the virus to undergo abortive versus latent or productive infection in such cells (Ahlqvist, et al. 2005).

In fact, HHV-6A and HHV-6B may represent different viruses with different pathogenic potential. HHV-6A is associated with severe disease more often than is HHV-6B. Yet, most studies suggest that HHV-6A is much less common than HHV-6B in the general U.S. population, with HHV-6A DNA being found in the blood of less than 1% of normal adults (in contrast to HHV-6B, which is believed to be present in essentially all adults). However, it is difficult to know the distribution of HHV-6A with certainty since there is presently no reliable serologic test that can distinguish HHV-6B from HHV-6A.

While little is known concerning the pathogenesis of HHV-6, multiple lines of evidence suggest a link between HHV-6 (predominantly HHV-6A) and multiple sclerosis (MS). These include reports of virus reactivation in patients with MS (Soldan, et al. 1997), an elevated prevalence of HHV-6A in such patients (Akhyani, et al. 2000), evidence of autoimmune cross-reactivity between HHV-6 reactive T cells and myelin basic protein (Tejada-Simon, et al. 2003), and an increase in virus DNA, mRNA and protein within MS plaques when compared to normal-appearing white matter from the same subject (Cermelli, et al. 2003; Challoner, et al. 1995; Goodman, et al. 2003; Opsahi, et al 2005). HHV-6 has also been implicated in the etiology of chronic fatigue syndrome (Josephs, et al. 1991; Kontaroff, 1988) and in some cases of epilepsy (Donati, et al 2003). Finally, HHV-6 is also a significant source of morbidity and mortality in the post-transplant setting, due to its ability to reactivate CMV infection and to cause encephalitis (Singh, et al. 2000; Zerr, et al. 2001) as well as bone marrow suppression.

### BRIEF SUMMARY OF THE INVENTION

In accordance with the purpose of this invention, as embodied and broadly described herein, this invention relates to compositions and methods for the detection and treatment of human herpesvirus (HHV)-6.

Additional advantages of the disclosed method and compositions will be set forth in pail in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows design and screening of siRNAs targeting HHV-6 U51 and gB. FIG. 1A shows four different siRNAs against HHV-6A U51 designed to target distinct regions of the U51 open reading frame (ORP); numbers refer to the first nucleotide position of each siRNA relative to the predicted translational start codon of the U51 ORF. HEK293 cells were cotransfected with an expression vector carrying an SV5 epitope-tagged, wild-type version of the HHV-6A U51 ORF (HHV-6A U51nco) plus either empty pcDNA3 plasmid, empty pSuppressorRetro (pSR) vector (si Vec), or pSR constructs containing the siRNA-carrying inserts indicated in the figure; the lane labeled “siNeg.Ctrl” corresponds to a pSR construct that contains an siRNA of irrelevant, sequence which has no homology in the human genome. The U51 expression construct and the various siRNA-carrying plasmids were added to cells at a 1:6 molar ratio and formulated with LIPOFECTAMINE-2000 reagent (Invitrogen, Carlsbad, Calif.). At 48 h posttransfection, cell lysates were prepared and analyzed by Western blotting with an SV5 epitope-specific antibody (upper panel); the band detected corresponds to a protein of approximately 30-kDa molecular mass (as expected for U51). The blot was then stripped and reprobed with a β-tubulin antibody to confirm equal loading (lower panel). FIG. 1B shows two different siRNA constructs against HHV-6A gB were designed and tested in a similar manner as for siU51. In this experiment, the gB expression plasmid vector pDisplay has an HA epitope tag. The blot shown was probed with an anti-HA antibody; the blot was then stripped and reprobed with a β-tubulin antibody to confirm equal loading (lower panel).

FIG. 2 shows suppression of U51 mRNA expression in virus-infected cell lines stably expressing siRNA-U51 (si6U51-812, SEQ ID NO:10; si6U5-130, SEQ ID NO:7). Stable SupT1 cells expressing the indicated siRNAs were generated following appropriate drug selection of cells transduced with corresponding retroviral vectors (pSuppressor Retro; Imgenex, San Diego, Calif.). The siRNA-expressing cells were then infected with HHV-6A (strain U1102) at an MOI of 0.1 TCID₅₀/cell, and total cellular RNA was extracted 24 h thereafter. Quantitative RT-PCR analysis was then performed, to assess levels of mRNA corresponding to U51. mRNA levels were normalized to GAPDH mRNA for each sample. Results represent mean values from a single experiment that was performed in triplicate (three independent infections); error bars correspond to the standard error of these mean values. There is a statistically significant difference in U51 mRNA levels in the siU51-SupT1 stable cell sample versus control SupT1 cells that were also infected with HHV-6 (P< 0.001; two-tailed t test).

FIG. 3 shows the effect of U51 knockdown on virus replication and syncytium formation. SupT1 cells stably expressing siRNA targeting U51, gB, or an irrelevant sequence (siNeg.Ctrl.) were infected with HHV-6A strain U1102 at an MOI of 0.1 TCID₅₀/cell. Virally induced cytopathic effects were then examined in the cultures at 6 days postinfection. The photomicrographs shown were taken on an Olympus IX81 microscope under bright-field illumination; final magnification is 10×. The various panels correspond, respectively, to (A) SupT1 cells expressing an irrelevant siRNA (siNeg.Ctrl.) or (B) a gB-specific siRNA (si6gB) as well as two different clonal SupT1 cell sublines, each of which expresses a U51-specific siRNA, (C) si6U51-812 and (D) si6U51-130. It can be readily appreciated that virally induced syncytium formation was greatly reduced in the SupT1 cells that expressed either the gB-specific siRNA or the two U51-specific siRNAs. (E) Cell-free supernatants were collected from virus-infected SupT1 cultures at 6 days postinfection, and virus genomic DNA in the supernatant was measured by quantitative DNA PCR analysis using primers and TAQMAN® (Roche Molecular Systems, Inc, Alameda, Calif.) probes specific for the HHV-6 U38 gene. The data shown are from the same samples as in panels A to D; the results are representative of three separate experiments. The viral DNA copy number in SupT1 cells stably expressing either si6AgB or si6U51 were both significantly different from the viral DNA copy number in control SupT1 cells that were also infected with HHV-6 (P<±0.05 for each pairwise comparison between the three experimental cell lines and the control SupT1 cells). The detection sensitivity of the assay is about 10 copies.

FIG. 4 shows expression of a codon-optimized form of U51 can restore virus replication in SupT1 cells mat express a U51-specific siRNA. SupT1 cells stably expressing an siRNA targeting U51 (si6U51-812) were transduced with a retrovirus vector that carried a human codonoptimized (CO) derivative of the HHV-6A U51 ORF. This CO version of the U51 ORF carries an mRNA that, is significantly different from the wild-type U51 mRNA at the nucleotide level, and as a result it is resistant to inhibition by the U51 siRNA. The SupT1(si6U51-812) cells and their CO-U51-transduced counterparts were then infected with HHV-6A strain U1102 at an MOI of 0.1 TCID₅₀/cell. Virally induced cytopathic effects were then examined in the cultures at 6 days postinfection, as described in the legend to FIG. 3. The various panels correspond, respectively, to SupT1 si6U51-812 cells transduced with the empty retrovirus vector (A), uninfected SupT1 cells (B), or CO-U51-encoding retrovirus vector encoding U51 from HHV-6A (C) or HHV-6B (D). It can be readily appreciated that virally induced syncytium formation was restored in the SupT1(si6U51-812) cells upon coexpression of CO-U51. FIG. 4E shows cell-free supernatants collected from virus-infected SupT1 cultures at 6 days postinfection, with virus genomic DNA in the supernatant measured by quantitative DNA PCR analysis, as described in the legend to FIG. 3E. The various lanes indicate control si6U51-812-expressing SupT1 cells and their CO-U51-transduced counterparts. The results are representative of three separate experiments. The viral DNA copy number in siU51-SupT1 stable cells transduced with either 6AU51co or 6BU51co were both significantly different from the viral DNA copy number in control siU51-SupT1 cells that were also infected with HHV-6 (P< 0.05 in both cases; two-tailed t test).

FIG. 5 shows virus infectivity unaffected by an antibody specific for U51. Two-hundred microliters of an HHV-6A virus stock (strain U1102) was preincubated with either 5 μl of human plasma (“baby plasma”) or 6 μg of affinity-purified rabbit antisera specific for HHV-6B U51 (anti-6B U51) or HHV-7 U51 (anti-7 U51) for 1 h at 37° C. The virus-antiserum mixture was then added to SupT1 cells (approximate MOI of 0.1 TCID₅₀/cell). Six days later, cell-free culture supernatants were collected and viral genomic DNA was measured by a quantitative PCR assay as previously described. The results are representative of three separate experiments. As expected, the human plasma efficiently neutralized HHV-6A infectivity (P< 0.05; two-tailed t test); in contrast, the U51-specific antisera had no such effect (P=0.117; two-tailed t test).

FIG. 6 shows tet-inducible overexpression of HHV-6B U51 in stably transduced HEK293 cells. HEK293 cells were cotransfected with die regulatory plasmid pcDNA6/TR and the inducible expression vector pcDNA4/TO (Invitrogen), which contained an insert sequence corresponding to the wild-type (non-codon-optimized) HHV-6B U51 sequence, with an added N-terminal SV5 epitope tag. Positive cell colonies were selected in the presence of 2 μg/ml blasticidin and 60 μg/ml zeocin for 3 weeks. Cell lysates from those positive cells, either in the absence of tetracycline treatment (“−”) or following induction with 1 μg/ml tetracycline for 24h (“+”), were prepared and analyzed by Western blot using an SV5-specific antibody (upper panel) or a β-tubulin antibody (lower panel). Protein expression for a representative cell clone is shown; this clone was used in the subsequent [³⁵S]GTPγS binding assay (Table 1).

FIG. 7 shows U51 enhances cell-cell fusion in the presence of VSV-G in vitro. Equal numbers of HEK293 cells were transfected either with a HIV-1 Tat expressing plasmid (pcTat) or with a plasmid containing a luciferase reporter gene under the transcriptional control of the HIV-1 LTR. All of the cells were also transfected with plasmid expression vectors encoding the following proteins: none (pcDNA3 lane), VSV-G alone (VSV-G lane) or VSV-G plus. HCMV US28, HHV-6A U51 (6AU51CO), or the rat kappa opioid receptor (KOR), which was included as a negative control The pcTat and LTRluc cell pools were then trypsinized 4 h posttransfection, mixed, and allowed to re-adhere to tissue culture plastic; 44 h later, luciferase activity was measured. The experiment shown is representative of three independent experiments. Shown are the mean relative light units (RLU) and standard deviations for three replicate samples obtained. As previously reported, HCMV US28 enhanced cell fusion initiated by US28 (P< 0.05). HHV-6A U51 had a similar, though slightly less pronounced, effect (P< 0.05), while KOR had no such effect (P=0.431; two-tailed t test).

FIG. 8 shows U11 sequence alignment, with key predicted epitopes underlined. The sequences shown are for strains Z29 (SEQ ID NO:185) and U1102 (SEQ ID NO:186).

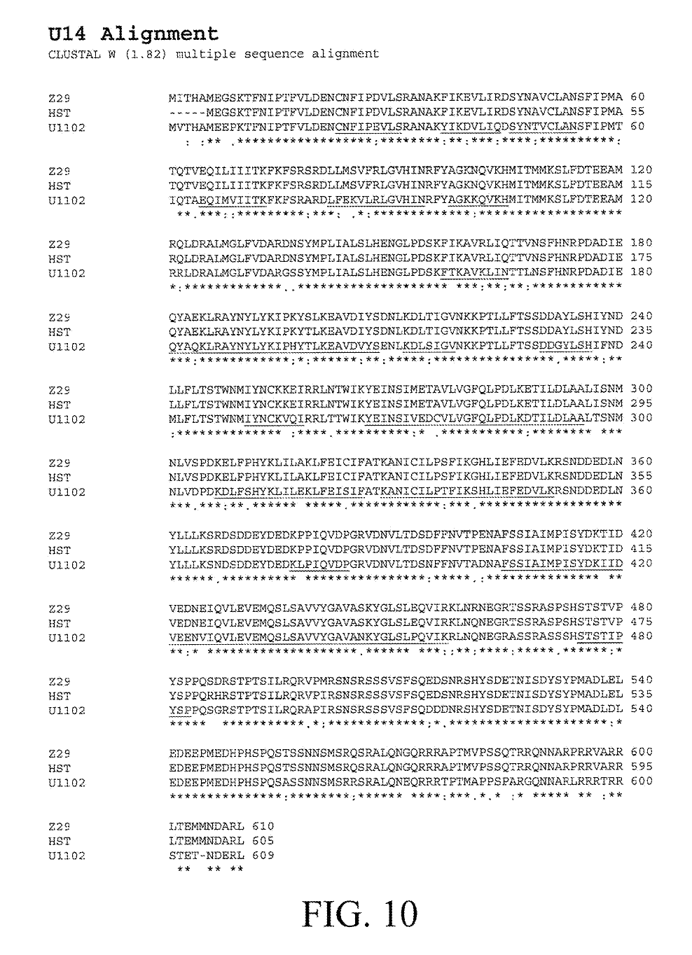

FIG. 9 shows U47 sequence alignment with key predicted epitopes underlined. The sequences shown are for strains GS (SEQ ID NO:187), U1102 (SEQ ID NO:188), and Z29/HST (SEQ ID NO:189).

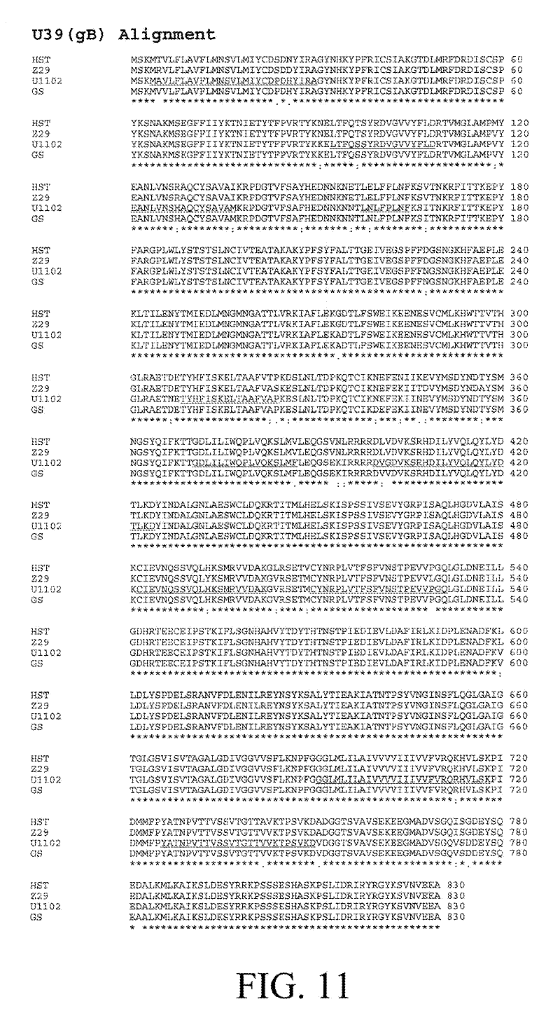

FIG. 10 shows U14 sequence alignment, with key predicted epitopes underlined. The sequences shown are for strains Z29 (SEQ ID NO:190), HST (SEQ ID NO:191), and U1102 (SEQ ID NO:192).

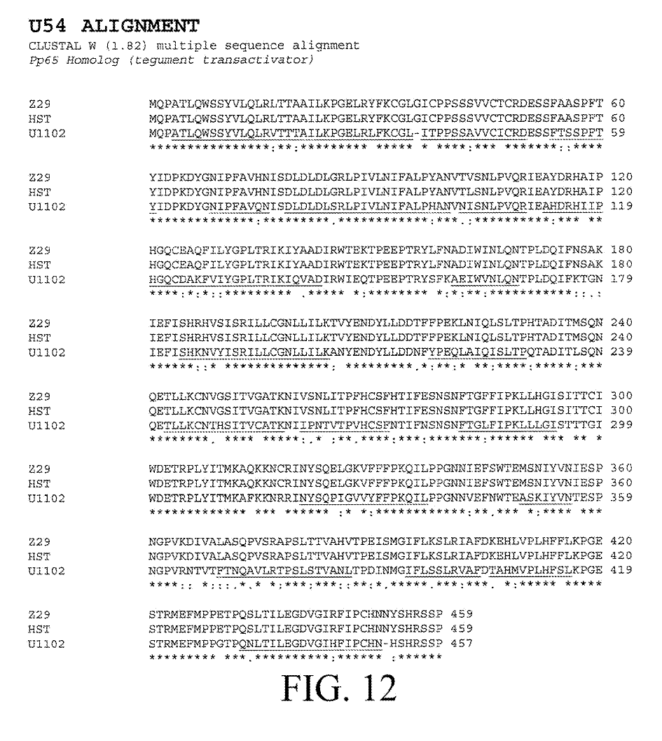

FIG. 11 shows U39 (gB) sequence alignment, with key predicted epitopes underlined. The sequences shown are for strains HST (SEQ ID NO:193), Z29 (SEQ ID NO:194), U1102 (SEQ ID NO:195), and GS (SEQ ID NO:196).

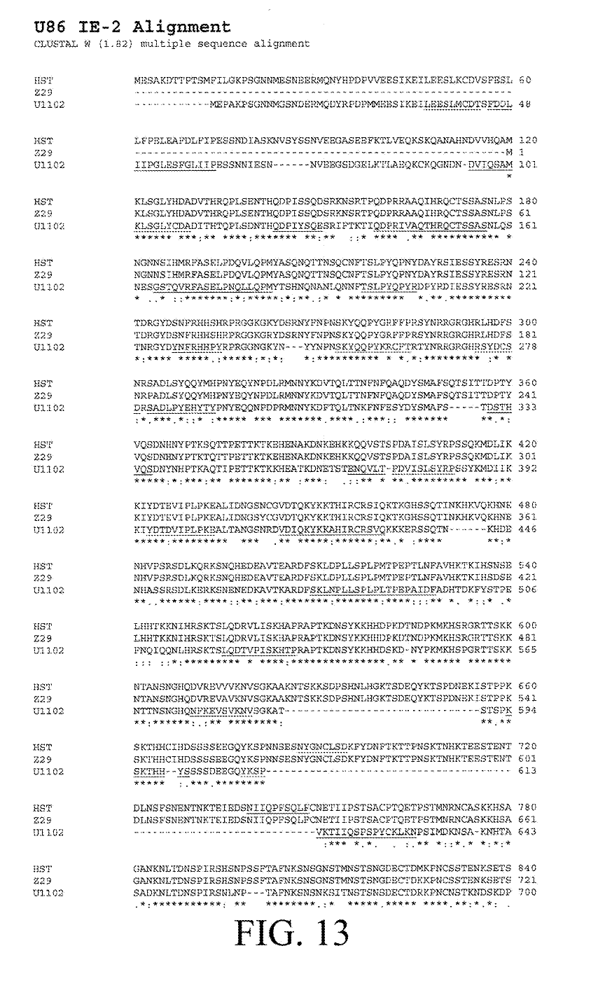

FIG. 12 shows U54 sequence alignment, with key predicted epitopes underlined. The sequences shown are for strains Z29 (SEQ ID NO:197), HST (SEQ ID NO:198), and U1102 (SEQ ID NO:199).

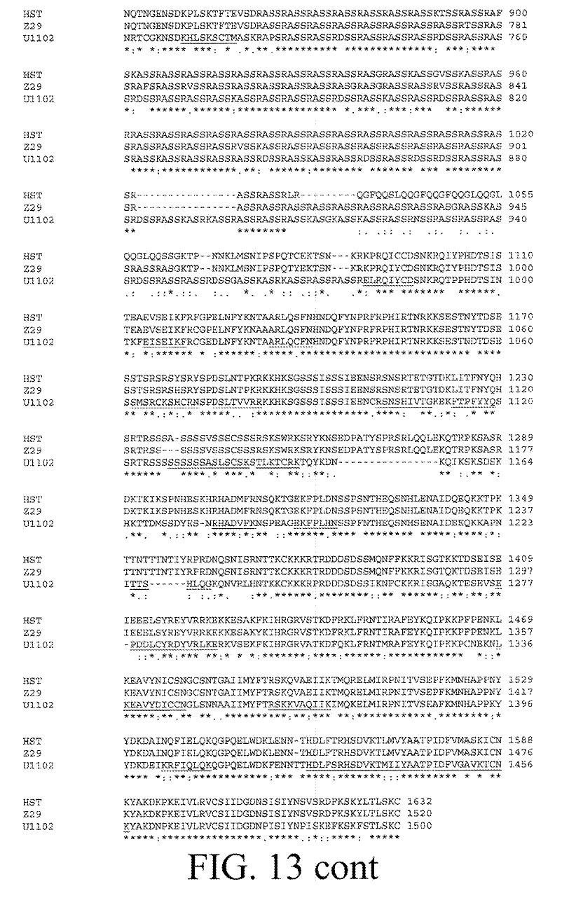

FIG. 13 shows U86 IE-2 sequence alignment, with key predicted epitopes underlined. The sequences shown are for strains HST (SEQ ID NO:200), Z29 (SEQ ID NO:201), and U1102 (SEQ ID NO:202).

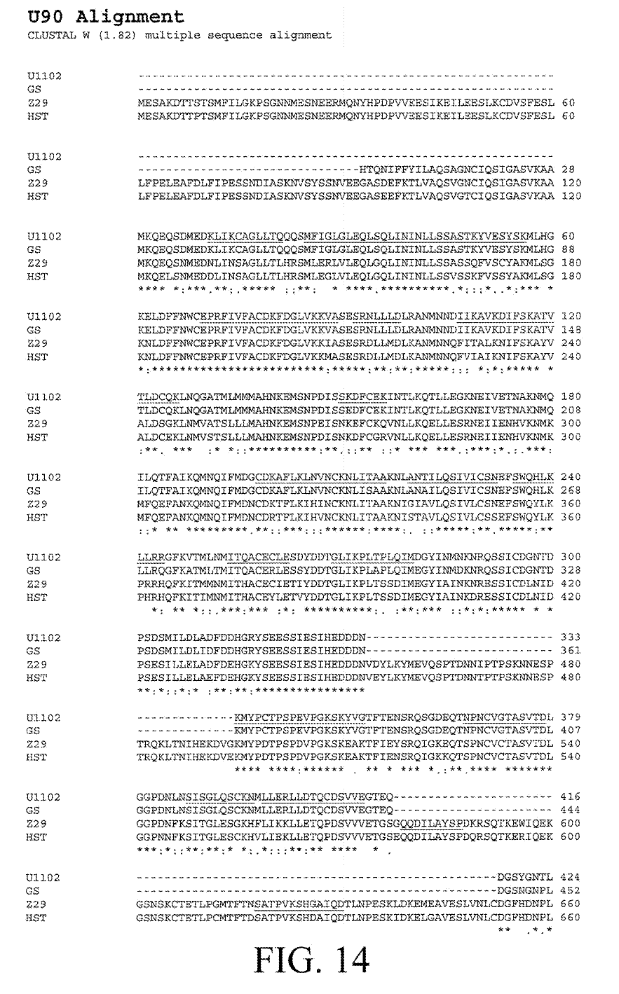

FIG. 14 shows U90 sequence alignment, with key predicted epitopes underlined. The sequences shown are for strains U1102 (SEQ ID NO:203), GS (SEQ ID NO:204), Z29 (SEQ ID NO:205), and HST (SEQ ID NO:206).

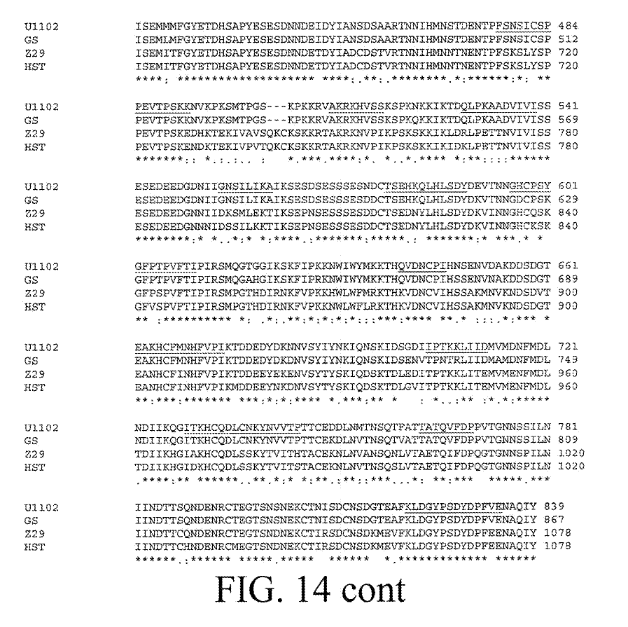

FIG. 15 shows alignment of proteases from HCMV and HHV-6. The sequences shown are for HHV-6A (SEQ ID NO:207), HCMV (SEQ ID NO:208), and HHV-7 (SEQ ID NO:209).

### DETAILED DESCRIPTION OF THE INVENTION

The disclosed methods and compositions may be understood more readily by reference to the following detailed description of particular embodiments and the Examples included therein and to the Figures and their previous and following descriptions.

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a polypeptide is disclosed and discussed and a number of modifications that can be made to a number of molecules including the polypeptide are discussed, each and every combination and permutation of polypeptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms a, an, and the include plural references unless the context clearly dictates otherwise. Thus, for example, reference to a polypeptide includes a plurality of such polypeptides, reference to tire polypeptide is a reference to one or more polypeptides and equivalents thereof known to those skilled in the art, and so forth.

Optional or optionally means that the subsequently described event, circumstance, or material may or may not occur or be present; and mat the description includes instances where the event, circumstance, or material occurs or is present and instances where it does not occur or is not present.

Ranges may be expressed herein as from about one particular value, and/or to about another particular value. When such a range is expressed, also specifically contemplated and considered disclosed is the range from the one particular value and/or to the other particular value unless the context specifically indicates otherwise. Similarly, when values are expressed as approximations, by use of the antecedent about, it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents, it will be clearly understood that, although a number of publications are referred to herein, such reference does not constitute an admission that, any of these documents forms part of the common general knowledge in the art.

Throughout the description and claims of this specification, the word comprise and variations of the word, such as comprising and comprises, means including but not limited to, and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the appended claims.

Provided herein is a method of treating or preventing HHV-6 infection in a subject, comprising administering to the subject a composition comprising an HHV-6 U51 (U51) inhibitor. The U51 gene is one of tire two 7-transmembrane (7-tm) receptors carried by HHV-6 (Gompels, et al. 1995). It has been shown to be most closely related to the UL78 gene family from human cytomegalovirus (CMV). HHV-6 U51 can bind certain CC-chemokines such as RANTES with nanomolar affinity (Beisser, et al. 1998).

As disclosed herein, U51 is involved in HHV-6 membrane fusion and chemokine sequestration. Thus, the U51 inhibitor of the present method can inhibit HHV-6 membrane fusion and/or chemokine sequestration by U51. The U51 inhibitor of the present method can be any known or newly identified composition that can modulate an activity of U51. Activities of a protein include, for example, transcription, translation, intracellular translocation, phosphorylation, stability, homophilic and heterophilic binding to other proteins, and degradation. Thus, the U51 inhibitor of the present method can inhibit gene expression of U51. The U51 inhibitor can also promote the cleavage of U51 by proteases or degradation of U51 by ubiquitin.

Also provided is a method of treating or preventing HHV-6 infection in a subject, comprising administering to the subject a composition comprising an HHV-6 glycoprotein gB (gB) inhibitor. As disclosed herein, gB is involved in HHV-6 attachment and membrane fusion with cell membranes. Thus, the provided method can inhibit HHV-6 binding and/or membrane fusion by inhibiting gB. The gB inhibitor of the provided method can be any known or newly identified composition that can modulate an activity of gB.

The U51 or gB inhibitor of the provided method can inhibit U51 or gB gene transcription. Thus, provided herein is a composition comprising a nucleic acid, wherein the nucleic acid inhibits expression of HHV-6 U51 or gB. Functional nucleic acids are nucleic acid molecules that have a specific function, such as binding a target molecule or catalyzing a specific reaction. Functional nucleic acid molecules include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of U51 or gB or the genomic DNA of U51 or gB or they can interact with the polypeptide encoded by U51 or with glycoprotein gB. Often functional nucleic acids are designed to interact, with other nucleic acids based on sequence homology between the target molecule and the functional nucleic acid molecule, hi other situations, the specific recognition between the functional nucleic acid molecule and the target molecule is not based on sequence homology between the functional nucleic acid molecule and the target molecule but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNase H (Ribonuclease H) mediated RNA-DNA hybrid degradation. Alternatively the antisense molecule is designed to interrupt a processing function that normally would take place on the target molecule, such as transcription or replication. Antisense molecules can be designed, based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437, which are incorporated herein in their entirety for such methods and techniques.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind very tightly with K_{d}S from the target molecule of less than 10⁻¹² M. It is preferred that the aptamers bind the target molecule with a K₄ less than 10⁻⁶, 10⁻⁸, 10⁻¹⁰, or 10⁻¹². It is preferred that the aptamer have a with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698, which are incorporated herein in their entirety for such methods and techniques.

Ribozymes are nucleic acid molecules that are capable of catalyzing a chemical reaction, either intramolecularly or intermolecularly. Ribozymes are thus catalytic nucleic acids. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat, which are incorporated herein in their entirety for such methods and techniques) hairpin ribozymes (for example, U.S. Pat. Nos. 5,631,115, 5,646,031, 5,683,902, 5,712,384, 5,856,188, 5,866,701, 5,869,339, and 6,022,962, which are incorporated herein in their entirety for such methods and techniques), and tetrahymena ribozymes (for example, U.S. Pat. Nos. 5,595,873 and 5,652,107, which are incorporated herein in their entirety for such methods and techniques). There are also a number of ribozymes that are not found in natural systems, but which have been engineered to catalyze specific reactions de novo (for example, U.S. Pat. Nos. 5,580,967, 5,688,670, 5,807,718, and 5,910,408, which are incorporated herein in their entirety for such methods and techniques). Preferred ribozymes cleave RNA or DNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756, which are incorporated herein in their entirety for such methods and techniques.

Triplex forming functional nucleic acid molecules are molecules that can interact with either double-stranded or single-stranded nucleic acids. When triplex molecules interact with a target region, a structure called a triplex is formed, in which there are three strands of DNA forming a complex dependant on both Watson-Crick and Hoogsteen base-pairing. Triplex molecules are preferred because they can bind target regions with, high affinity and specificity. It is preferred that the triplex tanning molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426, which are incorporated herein in their entirety for such methods and techniques.

External guide sequences (EGSs) are molecules that bind a target nucleic acid molecule forming a complex, and this complex is recognized by RNase P, which cleaves the target molecule. EGSs can be designed to specifically target a RNA molecule of choice, RNAse P aids in processing transfer RNA (tRNA) within a cell Bacterial RNAse P can be recruited to cleave virtually any RNA sequence by using an EGS that causes the target RNA: EGS complex to mimic the natural tRNA substrate. (WO 92/03566 by Yale, and Forster and Altaian, Science 238:407-409 (1990), which are incorporated herein in their entirety for such methods and techniques). Similarly, eukaryotic EGS/RNAse P-directed cleavage of RNA can be utilized to cleave desired targets within eukarotic cells, (Yuan et al., Proc. Natl. Acad, Sci. USA 89:8006-8010 (1992); WO 93/22434 by Yale; WO 95/24489 by Yale; Yuan and Airman, EMBO J 14:159-168 (1995), and Carrara et al., Proc. Natl. Acad. Sci. (USA) 92:2627-2631 (1995), which are incorporated herein in their entirety for such methods and techniques). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162, which are incorporated herein in their entirety for such methods and techniques.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al (1990) Plant Cell 2:279-89; Harmon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al (2001) Genes Dev., 15:188-200; Bernstein, E., et al (2001) Nature, 409:363-6; Hammond, S. M., et al (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al (2001) Cell, 107:309-21). At some point the siRNA duplex unwinds, and it appears that the antisense strand remains bound to RISC and directs degradation of the complementary mRNA sequence by a combination of endo and exonudeases (Martinez, J., et al (2002) Cell 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al., (2001) Nature, 411:494 498; Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), CheraGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for U51 or gB.

The production of siRNA from a vector is more commonly done through the transcription of a short, hairpin RNAs (shRNAs). Kits for the production of vectors comprising shRNA are available, such as, for example, Imgenex's GENESUPPRESSOR™ (Imgenex, San Diego, Calif.). Construction Kits and Invitrogen's BLOCK-IT™ (Invitrogen, Carlsbad, Calif.) inducible RNAi plasmid and lentivirus vectors. Disclosed herein are any shRNA designed as described above based on the sequences for the herein disclosed inflammatory mediators.

The nucleic sequence for U51, which represents nucleotides 82574 to 83479of the HHV-6 genome (see GenBank Accession No. NC_{—}001664) is set forth in SEQ ID NO:177. The target sequence for the disclosed functional nucleic acid (e.g., siRNA) can correspond to any nucleotide positions within SEQ ID NO:177. Provided is an siRNA comprising the nucleic acid sequence corresponding to nucleotide positions 130 to 148 within U51 or nucleotide positions 812 to 830 within U51. Thus, provided are siRNAs comprising the nucleic acid sequence set forth in SEQ ID NO:7or SEQ ID NO:10.

The nucleic sequence for glycoprotein gB (U39), which represents nucleotides 59588 to 62080 of the HHV-6 genome (see GenBank Accession No. NC_{—}01664) is set forth in SEQ ID NO:178. The target sequence for the disclosed functional nucleic acid (e.g., siRNA) can correspond to any nucleotide positions within SEQ ID NO:178. For example, provided is an siRNA comprising the nucleic acid sequence corresponding to nucleotide positions 861 to 879 within gB ORF. Thus, provided is an siRNA comprising the nucleic acid sequence set forth in SEQ ID NO:12.

Another activity of U51 that can be targeted is G-protein coupling. G-proteins belong to the larger grouping of GTPases. G-protein usually refers to the membrane-associated heterotrimeric G-proteins, sometimes referred to as the large G-proteins. These proteins are activated by G-protein coupled receptors and are made up of alpha (α), beta (β) and gamma (γ) subunits. There are also small G proteins or small GTPases like ras that are monomelic and not membrane-associated, but also bind OTP and GDP and are involved in signal transduction.

Receptor activated G-proteins are bound to the inside surface of the cell membrane. They consist of the G_{α} and the tightly associated G_{βγ} subunits. When a ligand activates the G-protein coupled receptor, the G-protein binds to the receptor, releases its bound GDP from the G_{α} subunit, and binds a new molecule of GTP. This exchange triggers the dissociation of the G_{α} subunit, the G_{βγ} dimer, and the receptor. Both G_{α}-GTP and G_{βγ} can then activate different ‘signaling cascades’ (or ‘second messenger pathways’) and effector proteins, while the receptor is able to activate the next G-protein. The G_{α} summit will eventually hydrolize the attached GTP to GDP by its inherent enzymatic activity, allowing it to re-associate with G_{βγ} and starting a new cycle. A well characterized example of a G-protein triggered signaling cascade is the cAMP pathway. The enzyme adenylate cyclase is activated by G_{αs}-GTP and synthesizes the second messenger cyclic adenosine monophosphate (cAMP) from ATP. Second messengers men interact with other proteins downstream to cause a change in cell behavior.

G_{α} subunits consist of two domains, the GTPase domain, and the alpha-helical domain. There exist at least 20 different alpha subunits, which are separated into several main families. G_{αs} or simply Gₛ (stimulatory) activates adenylate cyclase to increase cAMP synthesis. G_{αi} or simply Gᵢ (inhibitory) inhibits adenylate cyclase. G_{q} stimulates phospholipase C. G₀ stimulates K⁺ channels.

The β and γ subunits are closely bound to one another and are referred to as the beta-gamma complex. The G_{βγ} complex is released from the G_{α} subunit after its GDP-GTP exchange. The free G_{βγ} complex can act as a signaling molecule itself, by activating other second messengers or by gating ion channels directly. For example, the G_{βγ} complex, when bound to histamine receptors, can activate phospholipase A2. G_{βγ} complexes bound to muscarinic acetylcholine receptors, on the other hand, directly open G-protein coupled inward rectifying potassium (GIRK) channels.

The U51 inhibitor of the provided method can inhibit U51-mediated G-protein activation. Thus, the U51 inhibitor of the provided method can inhibit U51 coupling or uncoupling with G-protein. U51 exists in a constitutively active state preferentially coupled to G_{q/11}-proteins, which can be differentially redistributed to different G_{i/o}-proteins upon binding of different chemokines (Fitzsimons C P, et al. 2005). Specifically, CCL2, CCL5 and CCL11 act as agonists at U51, trafficking the receptor signal between G_{q/11}- and G_{i/o}-proteins (CCL5) or only to G_{i/o}-proteins (CCL2 and CCL11), whereas under non-stimulated conditions U51 constitutively signals mainly to G_{q/11}-proteins (Fitzsimons C P, et al. 2005). Thus, the U51 inhibitor of the provided method can inhibit U51 coupling or uncoupling with Gα_{q}, Gα₁₁, Gα_{q1}, Gα_{q2}, Gα_{q3}, or Gαₒ₁. The U51 inhibitor of the provided method can inhibit U51 activation of 0% G_{αq} Gα₁₁, Gα_{q1}, Gα_{q2}, Gα_{q3}, or Gαₒ₁.

G protein beta-gamma subunits associate with many binding partners in cellular signaling cascades. A peptide can cause G protein activation through a G_{βγ}-dependent, nucleotide exchange-independent mechanism. For example, the peptide SIGKAFKILGYPDYD (SEQ ID NO:210) forms a helical structure that binds the same face of G_{β1} as the switch II region of Gα (Davis T L, et al. 2005). SIRK can promote subunit dissociation by binding directly to G_{βγ} subunits and accelerating the dissociation of GαGDP without catalyzing nucleotide exchange. Thus, the U51 inhibitor of the provided method can bind G_{βγ} subunits. For example, the U51 inhibitor can comprise a polypeptide comprising the amino acid sequence set forth in SEQ ID NO:211, or an inhibitory fragment thereof.

The G-protein coupling inhibitor can also be a small peptide derivative of, peptidomimetic of, or small molecule ligand for conserved domains among G-protein coupled receptors (GPCRs).

The DRY motif is the single most highly conserved motif among G-protein coupled receptors (GPCRs) and is located close to the cytosolic surface of the third membrane-spanning domain (TM3). It is involved in interactions with the G protein or stabilization of the GPCR active conformation (Capra, V et al. 2004; Wess, J. 1998). Within the DRY motif, the most critical residue is the central, basic arginine. This is tightly conserved in almost all GPCRs. The acidic residue at position 1 is also very highly conserved, and is almost always either D or E (in 20% of cases) (Mirzadegan, T., et al 2003). The least conserved residue is the final Tyr (67% of cases), which is less important for receptor function (Mirzadegan, T., et al 2003). The DRY motif is well conserved in both 051 and U12 (it is present as ERI in TM3of HHV-6A and HHV-6B U51, and as IRY in TM3 of HHV-6A and HHV-6B U12, which represents a slightly divergent—but not unprecedented version of the DRY motif; (Mirzadegan, T., et al 2003)). In HCMV US28, mutation of the central Arg residue results in the loss of normal constitutive signaling activity, but no alteration in protein localization (Waldhoer, M., et al. 2003).

The NPXXY motif (SEQ ID NO:211) is also highly conserved among GPCRs and is located in the 7^{th} TM domain (Mirzadegan, T., et al. 2003). This motif is absent from U51, but present in the other viral GPCR-HHV-6A and HHV-6B U12 (as NPLVY, SEQ ID NO:212), and plays a role in tire transition of GPCRs from ground to activated state.

The cytoplasmic tail of the U51 and U12 is also conserved among GPCR. GPCR tails are often targets for GPCR-kinases (GRKs) that regulate intracellular trafficking and endocytosis of the receptors. In the case of HCMV US28, deletion of the cytoplasmic tail prevents normal constitutive endocytosis of the receptor (Waldhoer, M., et al. 2003), resulting in increased cell surface expression but no change in functional activity (Waldhoer, M., et al. 2003).

The G-protein coupling inhibitor can also be a polypeptide comprising an inhibitory fragment of U51, or a nucleic acid encoding said fragment, wherein the U51 fragment lacks all or part of the G-protein binding domain. Methods for determining the G-protein binding domain of a GPCR are known in the art and include, for example, protein crosslinking, co-immunoprecipitation, and X-ray crystallography.

Another activity of U51 or gB that can be targeted is ligand binding. Methods for inhibiting the binding of a protein to its receptor can, for example, be based on the use of molecules that compete for the binding site of either the ligand or the receptor.

Glycoprotein gB can bind cell surface heparan sulfate proteoglycans. Thus, the gB inhibitor can be heparin, a heparin analog or a synthetic derivative thereof or a molecule that blocks binding between gB and heparin.

U51 can bind certain β-chemokines such as RANTES. Thus, the inhibitor can be a derivative of a β-chemokine or a molecule that blocks binding between U51 and a chemokine. Non-limiting examples of β-chemokines include RANTES, monocyte chemoattractant protein 1 (MCP-1), and macrophage inflammatory protein 1α and 1β (MIP-1α and -1β). Thus, a ligand binding inhibitor can be, for example, a polypeptide that competes for the binding of a receptor without activating the receptor. Likewise, a ligand binding inhibitor can be a decoy receptor that competes for the binding of ligand. Such a decoy receptor can be a soluble receptor fragment (e.g., lacking transmembrane domain) or it can be a mutant receptor expressed in a cell but lacking the ability to transduce a signal (e.g., lacking cytoplasmic tail). Antibodies specific for either a ligand or a receptor can also be used to inhibit binding. The ligand binding inhibitor can also be naturally produced by a subject. Alternatively, the inhibitory molecule can be designed based on targeted mutations of either the receptor or the ligand.

Thus, as an illustrative example, the ligand binding inhibitor can be a polypeptide comprising a fragment of RANTES, wherein the fragment is capable of binding U51. The ligand binding inhibitor can further be a polypeptide comprising a fragment of U51. The fragment of U51 can lack the amino acids corresponding to the transmembrane domain.

Antibodies specific for U51 or a β-chemokine (e.g., RANTES) can be used to inhibit binding, for example, disclosed for use in the provided compositions and methods are neutralizing antibodies specific for U51, and nucleic acids encoding said antibodies. The term antibodies is used herein in a broad sense and includes both polyclonal, and monoclonal antibodies. In addition to intact immunoglobulin molecules, also useful in the methods taught herein are fragments or polymers of those immunoglobulin molecules. Also useful are human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods.

Administration of the antibodies can be done as disclosed herein. Nucleic acid approaches for antibody delivery also exist. The broadly neutralizing U51 antibodies and antibody fragments can also be administered to subjects as a nucleic acid preparation (e.g., DNA or RNA) that encodes the antibody or antibody fragment, such that the subject's own cells take up the nucleic acid and produce and secrete the encoded antibody or antibody fragment. The delivery of the nucleic acid can be by any means, as disclosed herein, for example.

The composition used in the herein provided methods can comprise an inhibitor of HHV-6 U51, an inhibitor of glycoprotein gB, or a combination thereof. The composition can further comprise inhibitors of other HHV-6 viral proteins. For example, the composition of the herein provided methods can further comprise an inhibitor of HHV-6 U94 gene product. The HHV-6 U94 gene product, which encodes a homolog of an adeno-associated virus (AAV) protein known as rep, can be involved in virus latency/reactivation or replication and is known to have DNA-binding activity. The nucleic sequence for U94, which represents nucleotides 141394 to 142866 of the HHV-6 genome (see GenBank Accession No. NC_{—}001664) is set forth in SEQ ID NO:179. Thus, a functional nucleic acid (e.g., siRNA) can target a sequence corresponding to nucleotide positions within SEQ ID NO:179.

The composition used in foe provided methods can further comprise an inhibitor of viral DNA polymerase. Examples of viral DNA polymerase inhibitors include ganciclovir, cidofovir, valacyclovir, foscarnet, and nucleoside or nucleotide analogs thereof.

The composition can further comprise an inhibitor of virally-encoded primase, helicase (U77) or other accessory proteins that are part of the viral DNA polymerase complex. The nucleic sequence for U77, which represents nucleotides 70823 to 73405 of the HHV-6 genome (see GenBank Accession No. NC_{—}001664, is set forth in SEQ ID NO:183. Thus, a functional nucleic acid (e.g., siRNA) can target a sequence corresponding to nucleotide positions within SEQ ID NO:183.

The composition used in the present methods can further comprise an inhibitor of viral ribonucleotide reductase. HHV-6 U28 encodes the large subunit of ribonucleotide reductase. The nucleic acid sequence for U28, which represents nucleotides 39020 to 41434 of the HHV-6 genome (see GenBank Accession No. NC_{—}001664), is set forth in SEQ ID NO:180. Thus, a functional nucleic acid (e.g., siRNA) can target a sequence corresponding to nucleotide positions within SEQ ID NO:180.

The composition can further comprise an inhibitor of HHV-6 encoded UL69 kinase (U42). The nucleic sequence for U42, which represents nucleotides 69054 to 70598 of the HHV-6 genome (see GenBank Accession No. NC_{—}001664), is set forth in SEQ ID NO:184. Thus, a functional nucleic acid (e.g., siRNA) can target a sequence corresponding to nucleotide positions within SEQ ID NO:184.

The methods described herein can include a composition comprising an inhibitor of HHV-6B U83 gene product. The product of the HHV-6B U83 gene, which is a highly selective CCR2 chemokine agonist, can bind to U51 and/or is involved in viral immune evasion or replication. The nucleic sequence for U83, which represents nucleotides 123528 to 123821 of the HHV-6 genome (see GenBank Accession No. NC_{—}501664), is set forth in SEQ ID NO:181. Thus, a functional nucleic acid (e.g., siRNA) can target a sequence corresponding to nucleotide positions within SEQ ID NO:181.

The composition used in the provided methods can further comprise an inhibitor of membrane fusion. The ability of the virus to trigger membrane fusion is important for cell-cell spread of virus. Fusion inhibitors can include structural mimics of receptors that are bound by virally-encoded glycoproteins such as gH, gL, gB, gQ and gO.

As used herein, the composition can further comprise an inhibitor of cyclooxygcnase (COX)-2. Inhibitors of COX-2 inhibit HCMV virus replication in cell culture. HHV-6 also activates COX-2 in certain cell types, indicating that inhibition of cellular COX2 can also suppress HHV-6 replication. Selective (e.g., celecoxib and rofecoxib) and non-selective (e.g., NSAIDS) COX-2 inhibitors are known in the art.

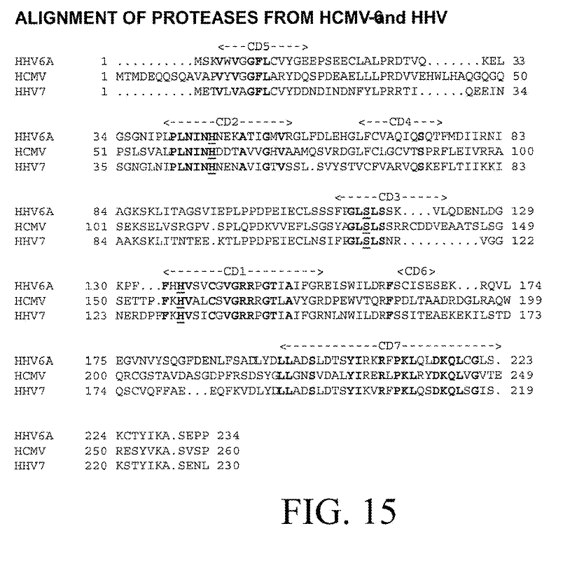

The composition used in the methods described herein can further comprise a HHV-6 protease blocker. Serine proteases found in other herpesviruses mediate essential proteolytic processing events during viral capsid maturation. All herpesviruses encode serine proteases with similar substrate—specificity (i.e., they all cleave a peptide bond between a serine and an alanine). These enzymes perform a proteolytic cleavage that is essential for virion maturation. These enzymes are catalytically inefficient compared with archetypal serine-proteases. They are also only weakly inhibited by common serine protease inhibitors such as phenylmethylsulphonyi fluoride (PMSF) and N-tosyl-L-Leucine chloromelhyl ketone (TLCK). These biochemical characteristics can be related to the unique structural properties of this class of enzymes, as exemplified by the novel polypeptide backbone fold and active site found in HCMV protease. Similarities between the HHV-6 and HCMV proteases include an overall level of 42% amino acid identity, as well as the conservation of the His-Ser-His catalytic triad, represented by amino acid residues His 63, Ser132, and His157 of HCMV protease, and by residues His46, Ser116 and His135 of the HHV-6 protease (see FIG. 15 for sequence alignment).

The composition used in foe herein provided methods can further comprise an inhibitor of viral alkaline nuclease. Deletion of UL12 (the gene which encodes alkaline nuclease in HSV-1) reduces virus production by 200-1000 fold. This gene is conserved in HCMV and in HHV-6 (U70). The nucleic sequence for U70, which represents nucleotides 105562 to 107028 of the HHV-6 genome (see GenBank Accession No. NC_{—}001664), is set forth in SEQ ID NO:182. Thus, a functional nucleic acid (e.g., siRNA) can target a sequence corresponding to nucleotide positions within SEQ ID NO:182.

The compositions of the provided method may be administered orally, rectally, intracisternally, intraventricular, intracranial, intrathecal, intra-articularly, intravaginally, parenterally (intravenously, intramuscularly, or subcutaneously), locally (powders, ointments, or drops), by intraperitoneal injection, transdermally, by inhalation or as a buccal or nasal spray. The exact amount of the therapeutic agent required will vary from subject to subject, depending on the age, weight and general condition of the subject, the severity of the disease that is being treated, the particular compounds used, the mode of administration, and the like. An appropriate amount may be determined by one of ordinary skill in the art using only routine experimentation given the teachings herein. Typical single dosages of therapeutic polypeptides such as antibodies range from about 0.1 to 10,000 micrograms, preferably between 1 and 100 micrograms. Typical polypeptide/antibody concentrations in a carrier range from 0.2 to 2000 nanograms per delivered milliliter.

Guidance for the therapeutic use of RNAi, including methods of administration and dosage, can be found, for example, in Murakami M. et al., Microbiol Immunol. 2005; 49(12): 1047-56; Carmona S. et al., Mol. Ther. 2006 February: 13(2):411-21; Leonard J N, Schaffer D V. Gene Ther. 2005 Sep. 22; Tompkins S M, Lo C Y, Tumpey T M, Epstein S L. Proc Natl Acad Sci USA. 2004 Jun. 8; 101(23):8682-6; and Giladi H. et al., Mol Ther, 2003 November; 8(5):769-76. For example, typical dosages of functional nucleic acids such as siRNA range from about 0.1 to 100 micrograms per kilogram (μg/kg), including between 1 and 50 μg/kg.

The compositions of the provided method may be administered as exogenous DNA (i.e., by gene transduction or transfection). Thus, the disclosed nucleic acids can be in the form of naked DNA or RNA, or the nucleic acids can be in a vector for delivering the nucleic acids to the cells, whereby the DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN™, LIPOFECTAMINE™ (GIBCO-BRL, Inc., Gaithersburg, Md.), SUPERFECT (Qiagen, inc. Hilden, Germany) and TRANSFECTAM™ (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a sonoporation machine (ImaRx Pharmaceutical Corp. Tucson, Ariz.).

As one example, vector delivery can be via a viral system, such as a retroviral vector system which can package a recombinant retroviral genome (see e.g. Fasten et al., Proc. Natl. Acad. Sci. U.S.A. 85:4486, 1988; Miller et al., Mol. Cell. Biol 6:2895, 1986). The recombinant retrovirus can then be used to infect and thereby deliver to the infected cells the nucleic acid. The exact method of introducing the nucleic acid into mammalian cells is, of course, not limited to the use of retroviral vectors. Other techniques are widely available for this procedure including the use of adenoviral vectors (Mitani et al., Hum. Gene Ther, 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther, 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g. s Remington: The Science and Practice of Pharmacy (19th ed.) ed. A. R. Gennaro, Mack Publishing Company, Eastern, Pa. 1995.

The composition can further comprise a pharmaceutically acceptable carrier. By pharmaceutically acceptable is meant a material that is not biologically or otherwise undesirable, which can be administered to an individual along with the selected substrate without causing significant undesirable biological effects or interacting in a deleterious manner with any of the other components of the pharmaceutical composition in which it is contained.

Depending on the intended mode of administration, the disclosed compositions can be in pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. The compositions will include an effective amount of the selected substrate in combination with a pharmaceutically acceptable carrier and, in addition, may include other medicinal agents, pharmaceutical agents, carriers, or diluents. Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglyeol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as, for example, starches, lactose, sucrose, glucose, mannitol, and silicic acid, (b) binders, as, for example, carboxymethylcellulose, alignates, gelatin, polyvinylpyrrolidone, sucrose, and acacia, (c) humectants, as, for example, glycerol, (d) disintegrating agents, as, for example, agar-agar, calcium carbonate, potato or tapioca starch, alginic acid, certain complex silicates, and sodium carbonate, (e) solution retarders, as, for example, paraffin, (f) absorption accelerators, as, for example, quaternary ammonium compounds, (g) wetting agents, as, for example, cetyl alcohol, and glycerol monostearate, (h) adsorbents, as, for example, kaolin and bentonite, and (i) lubricants, as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as, for example, ethyl alcohol, isopropyl alcohol, ethyl carbonate, ethyl acetate, benzyl alcohol, benzyl alcohol, benzyl benzoate, propyleneglyeol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

The term pharmaceutically acceptable salts, esters, amides, and prodrugs as used herein refers to those carboxylate salts, amino acid addition salts, esters, amides, and prodrugs of the compounds of the present invention which are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds of the invention. The terra salts refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, S. M, Barge et al., “Pharmaceutical Salts,” J. Pharm. Sci., 1977, 66:1-19 which is incorporated herein by reference.)

The present invention provides various methods of screening for inhibitors of HHV-6 replication and inhibitors identified by the screening methods. Thus, provided herein is a method of screening for an inhibitor of HHV-6 replication, comprising contacting with a candidate agent a cell comprising a nucleic acid encoding HHV-6 U51, wherein the nucleic acid is functionally linked to an expression control sequence, and detecting U51 gene expression in tire cell. A decrease in U51 expression indicates the candidate agent is an inhibitor of HHV-6 replication.

U51 gene expression can be detected using any suitable technique. Further, molecules that interact with or bind to U51, such as antibodies specific for U51, can be detected using known techniques. Many suitable techniques—such as techniques generally known for the detection of nucleic acids, proteins, peptides and other analytes and antigens—are known. In general, these techniques can involve nucleic acid amplification or hybridization, direct imaging (e.g., microscopy), immunoassays, or by functional determination. By functional determination is meant that a protein that has a function can be detected by the detection of said function. For example, an enzyme can be detected by evaluating its activity on its substrate.

U51 mRNA transcripts can be detected using standard, methods known in the art. Generally, these methods involve the use of sequence specific primers or probes for reverse transcription, amplification and/or hybridization. Primers and/or probes can be designed for any sequence given the information disclosed herein and known in the art. A variety of sequences are provided herein and these and others can be found in Genbank at www.pubmed.gov. Those of skill in the art understand how to resolve sequence discrepancies and differences and to adjust the compositions and methods relating to a particular sequence to other related sequences.

Immunodetection methods can be used for detecting U51 protein expression. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental immunology. Vol. 1: Immunochemistry, 27.1-27.20 (1986), each incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed widgets. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, flow cytometry, protein arrays, multiplexed bead arrays, magnetic capture, in vivo imaging, fluorescence resonance energy transfer (FRET), and fluorescence recovery/localization after photobleaching (FRAP/FLAP).

U51 gene expression can also be detected by functionally linking a marker gene to the U51 expression control sequence. Examples of marker genes include the E. coli lacZ gene, which encodes β-galactosidase, and green fluorescent protein (GFP).

The nucleic acid encoding HHV-6 U51 can be either endogenous to the cell or can be exogenously delivered to the cell. Nucleic acids can be delivered through a number of direct delivery systems such as electroporation, lipofection, calcium phosphate precipitation, plasmids, viral vectors, viral nucleic acids, phage nucleic acids, phages, cosmids, or via transfer of genetic material in cells or earners such as cationic liposomes. Such methods are well known in the art and readily adaptable for use with the compositions and methods described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

Nucleic acids that are delivered to cells typically contain expression controlling sequences. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273; 113 (1978)). The immediate early promoter of the human cytomegalovirus is conveniently obtained as a HindIII E restriction fragment (Greenway, P. J. et al., Gene 18:355-360 (1982)). Of course, promoters from the host cell or related species also are useful herein.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK). Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity of U51 should be employed whenever possible. When a crude extract is found to have a desired activity, further fractionation of the positive lead extract is necessary to isolate chemical constituents responsible for the observed effect. Thus, the goal of the extraction, fractionation and purification process is the careful characterization and identification of a chemical entity within the crude extract having an activity that stimulates or inhibits U51. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of U51.

Also provided herein is a method of screening for an inhibitor of HHV-6 replication, comprising contacting with a candidate agent a cell comprising a nucleic acid encoding HHV-6 U51 functionally linked to an expression control sequence and a nucleic acid encoding G-protein functionally linked to an expression control sequence and detecting calcium flux in the cell, a decrease in endogenous or ligand-activated calcium flux indicating the candidate agent is an inhibitor of HHV-6 replication.

Activation of certain sub-classes of GPCRs results in intracellular calcium mobilization. Calcium mobilization assays that make use of dyes that become highly fluorescent in the presence of calcium can be used. Calcium flux assays make use of dyes that become highly fluorescent in the presence of calcium. Examples of such dyes useful for the detection of calcium include, but are not limited to, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, fura-4F, fura-5F, fura-6F, fluo-4, fluo-5F, fluo-5N, Oregon Green 488 BAPTA, Calcium Green. Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Aequorin, dextran conjugates or any other derivatives of any of these dyes (Molecular Probes, Eugene, Oreg.). Methods for the detection of calcium flux are described in Nuccitelli, ed., Methods in Cell Biology, Volume 40: A Practical Guide to the Study of Calcium in Living Cells, Academic Press (1994): Lambert, ed., Calcium Signaling Protocols, Methods in Molecular Biology Volume 114, Humana Press (1999); and W. T. Mason, ed., Fluorescent and Luminescent Probes for Biological Activity. A Practical Guide to Technology for Quantitative Real-Time Analysis, Second Ed, Academic Press (1999), which are hereby incorporated herein by reference for their teaching of calcium flux assays.

Provided herein is a method of screening for an inhibitor of HHV-6 replication, comprising contacting with a candidate agent a cell comprising (1) a nucleic acid encoding HHV-6 U51, wherein the HHV-6 U51 encoding nucleic acid is functionally linked to an expression control sequence, and (2) a nucleic acid encoding G-protein, wherein the HHV-6 G-protein encoding nucleic acid is functionally linked to an expression control sequence, and detecting cell chemotaxis. A decrease in endogenous or ligand-activated chemotaxis indicates the candidate agent is an inhibitor of HHV-6 replication. Conventional chemotaxis assay procedures are known in the art. For example, according to one procedure, adherent cells are deposited onto one surface of an isoporous translucent polycarbonate membrane and placed within a solution containing a concentration gradient of pre-selected chemoattractant. The cells gradually move through the pores of a membrane, from one surface to another, towards or away from higher or lower concentrations of the chemoattractant. The cells form migrant and non-migrant cell populations on respective sides of the membrane. The cells are then manually scraped off one surface of the membrane, and the remaining cells on the other surface are labeled with a fluorescent compound and “counted” with a scanning fluorometer. Other chemotaxis assays have been described and can be found, for example, in U.S. Pat. Nos. 6,329,164, 6,238,874, 6,921,660, and 6,972,184, which are hereby incorporated herein by reference for their teaching of chemotaxis assays.

The present invention provides a method of screening for an inhibitor of HHV-6 replication, comprising contacting with a candidate agent a cell comprising (1) a nucleic acid encoding HHV-6 U51, wherein the HHV-6 U51 encoding nucleic acid is functionally linked to an expression control sequence, and (2) a nucleic acid encoding G-protein, wherein the HHV-6 G-protein encoding nucleic acid is functionally linked to an expression control sequence, and detecting U51 localization in the cell. A decrease in cell surface expression of U51 (or an increase in endocytosis) indicates the candidate agent is an inhibitor of HHV-6 replication, U51 cellular localization can be evaluated using standard methods known in the art, such as, for example, immunocytochemistry, flow cytometery, or cellular fractionation. Further, U51 can be functionally linked directly or indirectly (e.g., by secondary antibody) to a marker (e.g., GFP) such that the maker is detected by microscopy, flow cytometery, or cellular fractionation.

Also provided herein is a method of screening for an inhibitor of HHV-6 replication, comprising contacting with a candidate agent a cell comprising (1) a nucleic acid encoding HHV-6 U51, wherein the HHV-6 U51 encoding nucleic acid is functionally linked to an expression control sequence, and (2) a nucleic acid encoding G-protein, wherein the HHV-6 G-protein encoding nucleic acid is functionally linked to an expression control sequence, and detecting beta-arrestin translocation in the cell. A change in protein localization or a loss of ligand-induced association with U51 indicates the candidate agent is an inhibitor of HHV-6 replication. Upon agonist binding, G-protein coupled receptors (GPCRs) activate G proteins. The G_{α} and G_{βγ} subunits dissociate and the protein kinase, GRK, is recruited to the receptor at the plasma membrane. The GRK phosphorylates the carboxyl-terminal tail of the receptor. β-arrestin binds the GRK-phosphorylated receptor and uncouples the receptor from its cognate G protein. This process, termed desensitization, prevents over stimulation of the signaling cascade. β-arrestin then targets the desensitized GPCR to clathrin-coated pits where the receptor is internalized in clathrin-coated vesicles (CCV) and delivered to endosomes. β-arrestin dissociates from some receptors at or near the plasma membrane and is excluded from receptor-containing vesicles. In contrast, β-arrestin remains associated with other receptors and traffics with them into endocytic vesicles. Receptors that dissociate from β-arrestin at or near the plasma membrane are rapidly recycled whereas receptors that remain associated with β-arrestin are slowly recycled. Assay are available that utilize the redistribution of fluorescently-labeled arrestins from the cytoplasm to receptors at the plasma membrane to monitor the activation (or inactivation) of GPCRs. As an example, the TRANSFLUOR™ assay (Norak Biosciences, Research Triangle Park, N.C.) discriminates between agonists, partial agonists, and antagonists while providing valuable pharmacological information on efficacy and potency and is applicable to all GPCRs without requiring prior knowledge of natural ligands or how a given receptor is coupled to downstream signaling pathways.

Also provided herein is a method of screening for an inhibitor of HHV-6 replication, comprising contacting with a candidate agent a cell comprising (1) a nucleic acid encoding HHV-6 U51, wherein the HHV-6 U51 encoding nucleic acid is functionally linked to an expression control sequence, and (2) a nucleic acid encoding G-protein, wherein the HHV-6 G-protein encoding nucleic acid is functionally linked to an expression control sequence, and detecting G protein activation in the cell. A reduction in G protein activation indicates the candidate agent is an inhibitor of HHV-6 replication, Guanosine-γ-[³⁵S]thiotriphosphate ([³⁵S]GTPγS) can be used as an indicator of G protein activation as described in Example 1.

Further provided is a method of screening for an inhibitor of HHV-6 replication, comprising contacting a system comprising HHV-6 U5.1 and a β-chemokine with a candidate agent and detecting U51 binding to the β-chemokine. A reduction in binding as compared to a control indicates the candidate agent is an inhibitor of HHV-6 replication. Any known or newly discovered protein binding assay that can be used to detect U51 binding to the β-chemokine is disclosed herein. For example, immunoprecipitation of U51 can be combined with standard immunodetection techniques for the detection of the associated β-chemokine. Alternatively, the β-chemokine can be labeled with a detection marker or isotope, wherein the marker or isotope is used to detect the binding of β-chemokine on the cell surface. Other such methods are known and can be adapted for use in the herein method. Virtual or structural screening methodology can also be used to identify and rank U51 binding compounds on the basis of structural information.

The invention provides HHV-6 polypeptides, or fragments thereof, that can be used to detect antibodies to HHV-6. The polypeptides can be specific for antibodies to HHV-6B. Optimally, the polypeptides are specific for antibodies to HHV-6A. Thus, the polypeptide can be selected based on the identification of unique segments/regions within HHV-6A encoded proteins that diverge from the corresponding sequence in HHV-6B. Thus, HHV-6 polypeptides that can be used to detect antibodies to HHV-6 can be selected from within the HHV-7A or HHV-6B genome. The HHV-6A Genome (strain GS) can be found at Genbank Accession No. NC_{—}001664. The HHV-6B Genome (strain EST) can be found at Genbank Accession No. AB021506. The HHV-6B Genome (strain Z29) can be found at Genbank Accession No. NC_{—}00898. Sequences can further be selected on the basis that the proteins from which the peptides are derived are expected to be either highly abundant, immunodominant or both, and that the peptide domains have high potential for surface display and antibody recognition/immunogenicity. Examples of HHV-6 polypeptides that can be used to detect antibodies to HHV-6A and HHV-6B are provided in Tables 2-4. Also provided herein are polypeptides comprising the disclosed amino acid sequences, conservative variants, derivatives, and fragments thereof.

Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from tire protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PGR mutagenesis. Amino acid substitutions are typically of single residues, but can occur at a number of different locations at once; insertions usually will be on the order of about from 1 to 30 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally referred to as conservative substitutions.

Substantial changes in function or immunological identity are made by selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; (d) a residue having a bulky side chain, e.g., phenylalanine, is substituted for (or by) one not having a side chain, e.g., glycine, or (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or G-glycosylation (Ser or Thr). Deletions of cysteine or other labile residues also may be desirable. Deletions or substitutions of potential proteolysis sites, e.g., Arg, is accomplished for example by deleting one of the basic residues or substituting one by glutaminyl or histidyl residues.

Certain post-translational derivatizations are the result of the action of recombinant host cells on foe expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992): Ibba, Biotechnology & Genetic Engineering Reviews 13:197-216 (1995), Cahill et al, TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural, peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sri (1980) pp. 463-468; Hudson, D. et al., Int J Pept Prot Res 14:177-185 (1979) (—CH₂NH—, CH₂CH₂—) ; Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, BP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference). A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

Provided herein is a method for detecting antibodies to HHV-6 in a sample, comprising the steps of immobilizing an HHV-6 polypeptide on a surface; administering a sample, wherein HHV-6-specific antibodies in the sample bind the polypeptides; and detecting antibody bound to the polypeptides. Antibody detection methods are well known in the art and are described above. For example, bound antibody can be detected by measuring the amount of free polypeptide and subtracting from the total or by measuring bound polypeptide. Polypeptides could be labeled or labeled secondary antibodies could be used to detect the bound antibodies. The polypeptides could be anchored to a solid support (e.g., plate, bead, chip, array, slide, etc.).

Also provided herein is an HHV-6 antibody detection kit, comprising HHV-6 polypeptides, wherein the polypeptides are selected for being highly abundant, immunodominant, and bioavailable (high potential for surface display), and labeled anti-IgG antibodies.

The compositions disclosed herein and the compositions necessary to perform the disclosed methods can be made using any method known to those of skill in the art for that particular reagent or compound unless otherwise specifically noted.

For example, the nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et. al., Ann. Rev. Biochem. 53:323-356 (1984), (phosphotriester and phosphite-triester methods), and Narang et al. Methods Enzymol, 65:610-620 (1980), (phosphotriester method). Protein nucleic acid molecules can be made using known methods such as those described by Nielsen et al, Bioconjug. Chem. 5:3-7 (1994).

Polypeptides disclosed herein can be recombinant proteins obtained by cloning nucleic acids encoding the polypeptide in an expression system capable of producing the polypeptide. Expression systems for producing recombinant proteins are well known in the art and include adenovirus or baculovirus expression system. Optimally, genes inserted in viral and retroviral systems contain promoters and/or enhancers to help control the expression of the desired gene product. A promoter is generally a sequence or sequences of DNA that function when in a relatively fixed location in regard to foe transcription start site. A promoter contains core elements required for basic interaction of RNA polymerase and transcription factors, and may contain upstream elements and response elements.

Another method of producing the disclosed polypeptides is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc(tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides; A User Guide. W.H. Freeman, and Co., N.Y. (1992); Bodansky M and Trost B., Ed. (1993) Principles of Peptide Synthesis. Springer-Verlag Inc., NY, which are herein incorporated by reference at least for material related to peptide synthesis). Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994): Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Seimolzer, M et al. Science, 256:221 (1992)). This technique has been used to synthesize analogs of protein domains as well as large amounts of relatively pure proteins with full biological activity (deLisle Milton R C et al. Techniques in Protein Chemistry IV. Academic Press, New York, pp. 257-267 (1992)).

The following examples are set forth below to illustrate the methods and results according to the present invention. These examples are not intended to be inclusive of all aspects of the present invention, but rather to illustrate representative methods and results. These examples are not intended to exclude equivalents and variations of the present invention which are apparent to one skilled in the art.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Example 1

### The Human Herpesvirus 6 G Protein-Coupled Receptor Homolog U51 Positively Regulates Virus Replication and Enhances Cell-Cell Fusion

### Materials and Methods

Vector construction. The U51 wild-type genes (U51nco) were amplified by standard PCR methods. HHV-6A U51 was cloned from strain U1102. A simian virus 5 (SV5) epitope tag was introduced at the N terminus of U51, and KpnI-EcoRV restriction sites were added to facilitate cloning into the expression vector pcDNA3 (Invitrogen, Carlsbad, Calif.). The primer sets used for adding the SV5 tag was 5′-GAGGTACCGCCACCATGGAGGGCAAGCCCATCCCCAACCCCCTGCTGGGC CTGGACAGCACCGGAG-3′ (SEQ ID NO:1) and 5′-GGGCCTGGACAGCACCG GAGGCGGCAGCAAAGAAACGAAGTCTTTGGCT-3′ (SEQ ID NO:2).

The human codon-optimized (CO) U51 genes were assembled from synthetic oligonucleotides and cloned into pPCRScript (Geneart, Regensburg, Germany), as previously described (Bradel-Tretheway, et al. 2003). Note that the amino acid sequences encoded by these CO genes are identical to their wild-type counterparts (Bradel-Tretheway, et al. 2003). HHV-6A U51co was then restricted with HindIII and ApaI and cloned into pLEGFP-N1 renoviral vector (Clontech, Mountain View, Calif.).

A truncated version of HHV-6A gB without the putative N-terminal signal peptide and C-terminal transmembrane region (nucleotide positions 23 to 652) was amplified from the corresponding HHV-6A strain U1102 cosmid DNA clone (Neipel, et al. 1991) and then inserted at the SmaI-PstI sites of pDisplay plasmid vector (Invitrogen, Carlsbad. Calif.), which contains a signal peptide and a hemagglutinin (HA) epitope tag at the N terminus and a platelet-derived growth factor receptor transmembrane domain at the C terminus. The following primer sets were used for amplification: 5′-TACCCGGGAGATCTCCGGATCATTATATCAGAGCGCGCTA-3′ (SEQ ID NO:3) and 5′-CGCTGCAGAGAATTAATCCCATTAACATACGAAGGTG-3′ (SEQ ID NO:4).

To construct the 19- to 21-nucleotide hairpin siRNA cassettes, two cDNA oligonucleotides were chemically synthesized, annealed, and inserted between the SalI (XhoI) and XbaI sites immediately downstream of the U6 promoter in pSuppressorRetro vector (Imgenex, San Diego, Calif.): 5′-TCGA-19 nt-AACG-19 nt-TTTTT-3′ (SEQ ID NO:5) and 5′-CTAGAAAAA-19 nt-CGTT-19 nt-3′ (SEQ ID NO:6). The target sequences for each of the genes were as follows: si6U51-130, 5′-GTCGGTCGAGAATACGCTGTG-3′ (SEQ ID NO:7), corresponding to nucleotide positions 330 to 148 within the U51 open reading frame (ORE); si6U51-336, 5′-GAATACGCTGTGTTTACAT3, (SEQ ID NO:8), corresponding to nucleotide positions 136 to 154; si6U51-646, 5′-ATAGCGCATCTGCCGAA AG-3′ (SEQ ID NO:9), corresponding to nucleotide positions 646 to 664; si6U51-812, 5′-GTATCTGGCTGGTCAATTT-3′ (SEQ ID NO:10), corresponding to nucleotide positions 832 to 830; si6U51-812Scramble, 5′-ACGCGTATTGTCTATTTGG-3′ (SEQ ID NO:11), corresponding to a randomly arranged (scrambled) version of the sequences corresponding to nucleotide positions 812 to 830; and si6gB-A861, 5′-ATCGGTGTGTATGCTAA AG-3′ (SEQ ID NO:12), and si6gB-B1517, 5′-GTGAAACGATGTGTTATAA-3′ (SEQ ID NO:3), corresponding to nucleotide positions 863 to 879 and 1517 to 1535 within the gB ORF, respectively. A similar vector containing an irrelevant sequence that does not show significant homology to any human gene sequence (Imgenex) was used as a negative control (siNeg.Ctrl.; 5′-tcgaTCAGTCACGWAATGGTCGTrttcaagagaAACGACCATTAACGTGACTGAtt ttt-3′ (SEQ ID NO:14) and 5′-ctagaaaaaTCAGTCACGTTAATGGTCGTTtctettgaaAACGACCATTAACGTGACT GA-3′ (SEQ ID NO:15); nucleotides in uppercase letters represent stem structure of siRNA).

The knockdown efficiency of each siRNA construct was tested by cotransfecting the corresponding DNA plasmid into human embryonic kidney 293 (HEK293) cells together with a U51- or gB-expressing plasmid (as appropriate). Forty-eight hours after translation, protein expression levels were assessed by Western blotting.

Antibodies and Western blotting. Mouse monoclonal antibodies to the SV5 (paramyxovirus SV5, simian virus 5) or HA (hemagglutinin) epitopes and β-tubulin were purchased from Serotec (Raleigh, N.C.; MCA1360P) and Santa Cruz Biotech. (Santa Cruz, Calif.; sc-7392 and sc-9104), respectively.

For Western blotting, HEK293 cells were lysed with radioimmunoprecipitation assay buffer (Upstate, Charlottesville, Va.) and then mixed with loading buffer containing 200 mM. 2-mercaptoethanol without heating. Protein concentration was measured by a Bradford assay. Equal amounts of protein (25 μg) were loaded per lane and separated by sodium dodecyl sulfate—12% polyacrylamide gel electrophoresis prior to transfer to nitrocellulose. After incubation with appropriate primary antibodies (above) and washing, anti-rabbit or anti-mouse immunoglobulin G conjugated with horseradish peroxidase (Amersham Biosciences, Piscataway, N.J.) was then added. The blot was developed with enhanced chemiluminescence reagent (Amersham Biosciences, Piscataway, N.J.) and quantitated by National Institutes of Health Image software.

Retrovirus generation. HEK-293T cells were cotransfected with 5 μg of retrovirus vector plasmid (containing the siRNA of interest in pSuppressor or HHV-6A U51-CO in pLEGFP-N1) plus 5 μg p10A1 or pVSV-G, respectively, in a 100-mm culture dish by using the LIPOFECTAMINE™ transfection method. The culture medium was replaced 16 h later, and the viruses were collected from the culture supernatants 48 h posttransfection. For U51 add-back experiment, the retroviruses expressing HHV-6A U51-CO were concentrated by centrifugation of the virus supernatant at 50,000×g for 90 min at 4° C., and the pellet was then resuspended in 1% of the original volume in TNE (50 mM Tris-HCl [pH 7,8], 130 mM NaCl, 1 mM EDTA) buffer. Titers for the U51-CO expression constructs were about 10⁷ CFU/ml.

Viruses and cells: preparation of HHV-6 virus stocks. The U1102 strain of HHV-6A was used throughout this study. JJhan cells infected with HHV-6A were cocultivated with uninfected cells at a ratio of 1:13 for 7 days. Virus stocks were prepared by centrifugation of the culture fluids at 2,000×g for 10 min. and the supernatant was stored at −80° C. The 50% tissue culture infectious dose (TCID50) was calculated using the Spearman-Karber formula. SupT1 cells were maintained in RPMI 1640 containing 10% fetal calf serum, 100 U/ml penicillin, and 100 μg/ml streptomycin at 37° C. in a 5% CO₂ incubator.

Virus infection: retrovirus transduction and generation of a stable siRNAexpressing cell line. SupT1 cells were transduced with siRNA-expressing retrovirus supernatant at a 1 to 2 dilution in the presence of 6 μg/ml polybrene (Sigma, St. Louis, Mo.). Supernatant was removed after 24 h and replaced with fresh growth medium. Forty-eight hours after transduction, cells were passaged and selected for stable transform ants in medium containing geneticin (1,000 μg/ml). Three weeks after selection, cell colonies that were resistant were transferred to 96-well plates and expanded. Cells (5×10⁵) were mixed with 200 μl virus preparation at a multiplicity of infection (MOI of 0.1 TCID₅₀/cell, and virus was then centrifugally adsorbed onto the cells to enhance the efficiency of infection (2,000×g, 30 min). The infected cells were then washed once and suspended in 10 ml RPMI 1640 medium containing 10% fetal calf serum.

RNA extraction and real-time PCR. Total RNA was prepared from SupT1 cells that had been infected with HHV-6 by using High Pure RNA Isolation kits (Roche, Nutley, N.J.). Primer extension reactions were performed with SUPERSCRIPT™ II Firststrand cDNA Synthesis kits (Invitrogen, Carlsbad, Calif.) using oligo(dT) primer, in accordance with the manufacturer's instructions. mRNA expression levels of each gene were quantitated by TAQMAN® (Roche, Basel, Switzerland) real-time reverse transcription-PCR (RT-PCR) using U51-specific primers and probe and normalized with GAPDH mRNA. The U51-specific primer set was 5′-CCAAGGCTCTGGCAAAGGT-3′ (SEQ ID NO:16; sense) and 5′-TCAGCATCTGAAGAGCTTGCA-3′ (SEQ ID NO:17; antisense). The TAQMAN® (Roche, Basel, Switzerland) probe used was 5′-TTTCCCGATAGTTTGGATCATA-3′ (SEQ ID NO:18). GAPDH primers and probes (ASSAY-ON-DEMAND REAGENT™) were obtained from a commercial supplier (Applied Biosystems, Inc. (ABI), Foster City, Calif.).

Real-time quantitative DNA-PCR. The viral DNA load in HHV-6A U1102-infected cells was quantitated by TAQMAN® (Roche, Basel, Switzerland) real-time PCR. The HHV-6A U38 polymerase gene was chosen as a target gene for this purpose, and primer sets used for amplification of U38 were 5′-TGCTTCTGTAACGTGTCTTGGAA-3′ (SEQ ID NO:19: sense) and 5′-TCGGACTGCATCTTGGAATTAA-3′ (SEQ ID NO:20; antisense). The TAQMAN® (Roche, Basel, Switzerland) probe used was 5′-ATGCTTTGTTCCACGGTGGAT-3′ (SEQ ID NO:21). A standard curve for U38 DNA quantitation was generated by using serially 10-fold-diluted plasmid DNA containing the relevant gene sequence. Culture supernatants of virally infected cells were treated with Proteinase K, and DNA was extracted using WIZARD® DNA extraction kits (Promega, Madison, Wis.). This was used as the template and was analyzed with a Bio-Rad ICYCLER® (Bio-Rad, Hercules, Calif.). Amplification of standard and sample DNAs was conducted in the same 96-well reaction plate (Bio-Rad, Hercules, Calif.) under the following conditions: 2 min at 50° C. and 10 min at 95° C., followed by 50 cycles of 95° C. for 15 s and 60° C. for 1 min. The detection limit, is about 10 copies/reaction. All standards and samples were assayed in triplicate.

Neutralization assay. The U51-specific antiserum used was a polyclonal rabbit antiserum directed against HHV-6B U51 (raised against a purified synthetic peptide spanning the third predicted extracellular loop of HHV-6B U51[CHLPKAALSEIESDK (SEQ ID NO:22)]; there is only a single amino acid difference between HHV-6A and -6B within this region, which is denoted by the underlined residue; note that this same peptide was previously used by Menotti and colleagues to generate a U51-specific antiserum in rabbits [Menotti, et al. 1999]). The 15mer peptide was synthesized and injected into rabbits for antibody production. After affinity purification using a peptide-conjugated column, the purified antibody was able to detect both HHV-6A and HHV-6B U51 effectively (down to a dilution of 1:1,000) in an indirect immunofluorescent assay on virus-infected cell cultures. Purified U51 antiserum was incubated with 200 μl of HHV-6A U1102 virus supernatant in a total volume of 500 μl at 37° C. for 1 h. After that, infection was performed as described above. Note that the antiserum was not heat inactivated and thus would have been expected to be capable of mediating complement-directed lysis of virus particles in the event that complement-fixing antibodies were bound to cell-free virions.

Opioid receptor binding assay. To determine if HHV-6B U51 bound opioids, CHO-CAR cells were infected with a recombinant adenovirus that expressed the human codon-optimized HHV-6B U51 open reading frame (HHV6BCOwt) using methods previously described (Zhen, et al. 2004). Membranes from these cells were then prepared and incubated with opioids that were selective for the μ ([³H]DAMGO, 5 nM), δ ([³H]naltrindole, 1 nM; [³H]DPDPE, 10 nM), and κ ([³H]U69,593, 5 nM; [³H]bremazocine) receptors. Also, the nonselective antagonist [³H]diprenorphine was tested to determine if HHV-6B U51 would bind this nonselective high-affinity opioid. Nonspecific binding was measured by the inclusion of either 10 μM naloxone or 10 μM of the unlabeled compound. After a 60-min incubation, binding was terminated by filtering the samples through Schleicher & Schuell no. 32 glass fiber filters (Schleicher & Schuell, Keene, N.H.) using a Brandel. 48-well cell harvester. Filters were soaked for at least 60 min in 0.25% polyethylenimine for [³H]naltrindole and [³H]U69,593 binding experiments. After filtration, filters were washed three times with 3 ml of cold 50 mM Tris-HCl, pH 7.5, and were counted in 2 ml of ECOSCINT™ A (National Diagnostics, Atlanta, Ga.) scintillation fluid.

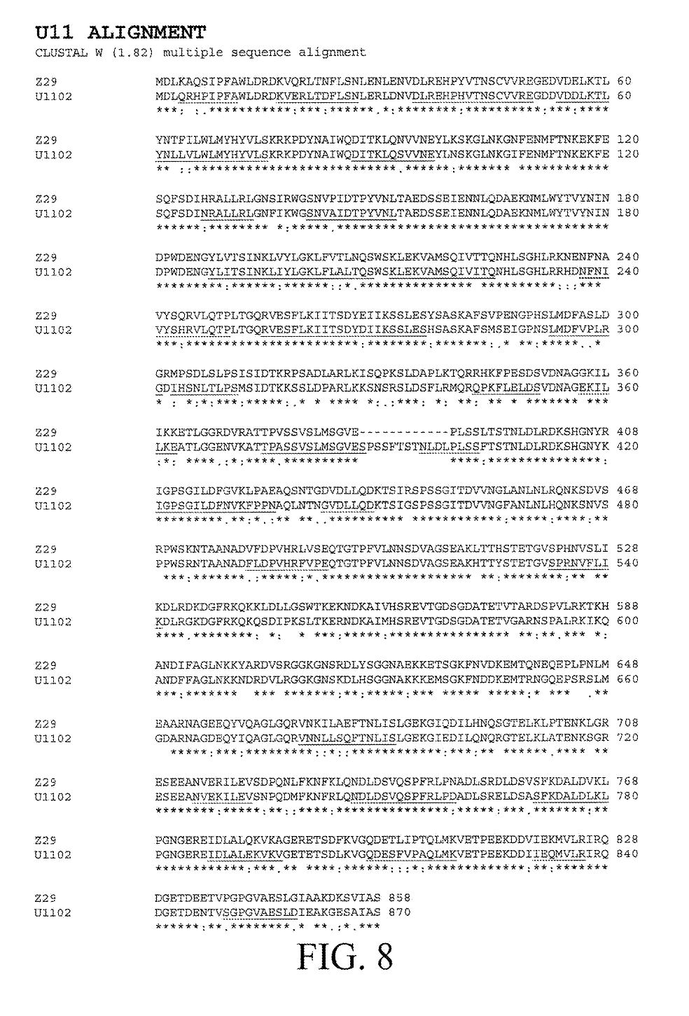

Establishment of a Tet-inducible cell line expressing U51. The T-Rex expression system (Invitrogen) was used to create a HEK293 cell line that inducibly expressed U51 upon addition of tetracycline (Tet). To do this, the native (noncodon-optimized) HHV-6B U51 open reading frame bearing an N-terminal. SV5 tag (Bradel-Tretheway, et al. 2003) was excised from a parental pcDNA3 vector with KpnI and EcoRV and inserted into pcDNA4/TO. pcDNA4/TO-U51 was then cotransfected with the pcDNA6/TR regulatory vector in a 1:6 ratio into HEK293 cells. After 48 h, cells were selected with 2 μg/ml biasticidin and 60 μg/ml ZEOCIN™ (Invitrogen, Carlsbad, Calif.). Selection of subclones for use in future experiments was based upon the induction profile of U51 expression following treatment of cells with tetracycline, as assessed by Western blot and flow cytometric analyses (representative results for one highly inducible subline are shown in FIG. 6). Cells treated for 24 to 48 h with Tet showed optimal U51 expression.

[³⁵S]GTPγS binding assay to measure coupling to G proteins. Three different sets of HEK293 cell membranes were used in experiments, including those from native cells and cells stably transfected with a Tet-inducible mammalian expression plasmid (Invitrogen, Carlsbad, Calif.) encoding an SV5 epitope-tagged derivative of the HHV-6B U51 protein (note that this construct was based on the native, noncodon-optimized viral sequence encoding U51). The latter cells were examined both in their native, uninduced state (in which U51 was expressed at a low level) and following induction (1 μg/ml tetracycline for 24 h), which resulted in a roughly 50- to 100-fold upregulation of U51 expression at both the RNA and protein levels (as measured by quantitative RT-PCR analysis as well as immunoblot analysis and flow cytometry, see FIG. 6).

Cells were scraped from tissue culture plates and then centrifuged at 1,000×g for 10 min at 4° C. The cells were resuspended in phosphate-buffered saline, pH 7.4, containing 0.04% EDTA. After centrifugation at 1,000×g for 10 min at 4° C., the cell pellet was resuspended in membrane buffer, which consisted of 50 mM Tris-HCl, 3 mM MgCl2, and 1 mM EGTA, pH 7.4. The membranes were vortexed, followed by centrifugation at 40,000×g for 30 min at 4° C. The membrane pellet was resuspended in membrane buffer, and the centrifugation step was repeated. The membranes were then resuspended in assay buffer, which consisted of 50 mM Tris-HCl, 3 mM MgCl₂, 100 mM NaCl, and 0.2 mM EGTA, pH 7.4. The protein concentration was determined by the Bradford assay (Bradford 1976) using bovine serum albumin as the standard. The membranes were frozen at −80° C. until use. HEK293 cell membranes as described above (15 μg of protein per tube) were incubated with 11 different ligands (ICI, 1 μM; RANTES 100 ng/ml; MCP-3, 1 ng/ml; lymphotactin, 100 ng/ml; interleukin-8, 100 ng/ml; the μ-opioid morphine, 1 μM; the δ-selective peptide DPDPE, 1 μM; the δ-selective antagonist naltrindole, 1 μM; and the μ-selective peptide DAMGO, 1 μM) in assay buffer for 60 min at 30° C. in a final volume of 0.5 ml. The reaction mixture contained 3 μM GDP and 80 pmol of [³⁵S]GTPγS. Basal activity was determined in the presence of 3 μM GDP and in the absence of an agonist, and nonspecific binding was determined in the presence of 10 μM unlabeled GTPγS. The membranes were then filtered onto glass fiber filters by vacuum filtration, followed by three washes with 3 ml of ice-cold 50 mM Tris-HCl, pH 7.5. Samples were counted in 2 ml of ecoscint A scintillation fluid. Data represent the percent of agonist stimulation [³⁵S]GTPγS binding of the basal activity, defined as (specific binding/basal binding)×100. All experiments were repeated at least three times and were performed in triplicate.

Cell fusion assay. A cell fusion assay was devised, which relies upon the expression of a transcriptional activator protein (HIV-1 Tat) in one population of cells and the presence of a transcriptional reporter for Tat in a second population of cells (a plasmid containing tire luciferase reporter gene, placed under the transcriptional control of the HIV-1 long terminal repeat [LTR], was used for this purpose). When the two populations of cells fuse, Tat will activate the HIV-1 LTR, resulting in high levels of luciferase production.

The fusion assay was performed by transfecting equal numbers of subcontinent HEK293 cells with either a HIV-1 Tat expressing plasmid (pcTat) (Tiley, et al. 1992) or an HIV-1 LTR:luciferase plasmid (Dollard, et al. 1994). All of the cells were also transfected with plasmid expression vectors encoding potential fusion-inducing proteins of interest. These were the VSV-G protein (pVSV-G; 0.3 μg; Clontech) and various 7-transmembrane proteins (human cytomegalovirus [HCMV] US28, the rat kappa opioid receptor, or HHV-6 U51). Four hours after transfection, the two populations of cells (Tat+ and LTR+) were treated with 0.25% trypsin-EDTA and mixed at a 1:1 ratio prior to reseeding in 12-well plates. Forty-four hours thereafter, luciferase assays were performed using commercially available reagents (Promega). Luciferase activity was quantitated with a Packard LUMICOUNT® (Packard, Meridan, Conn.) microplate luminometer within the linear range of the detector. Results are presented as relative light units.

### Results

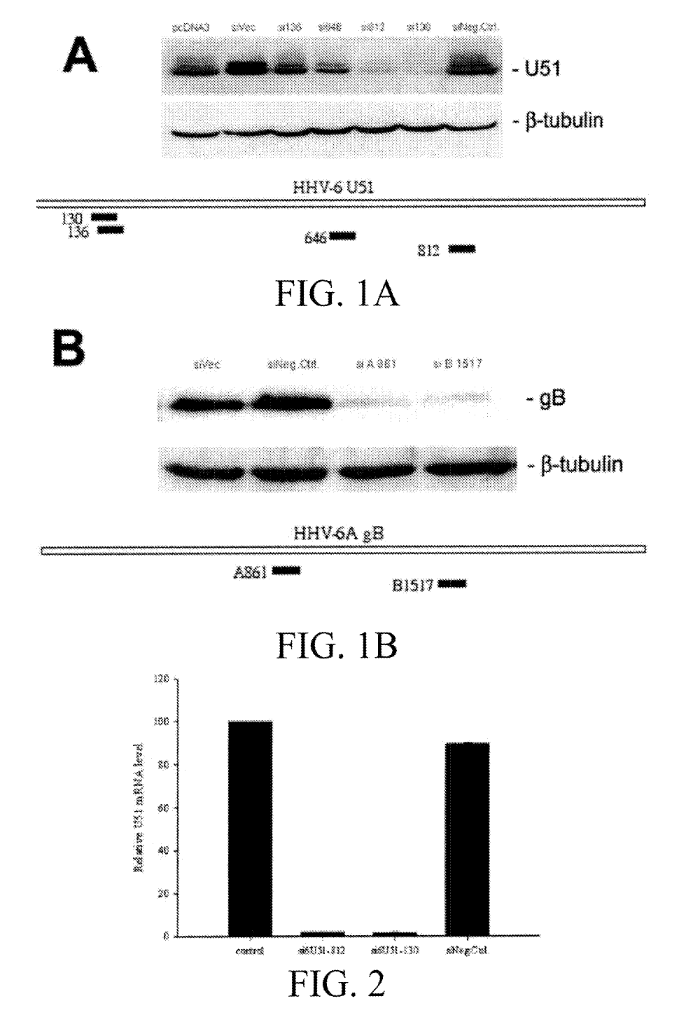

siRNA-expressing vectors suppressed U51 protein levels in a transient transfection system. RNA interference technology was used as a means to examine the functional importance of the U51 open reading frame (ORF) in the in vitro replication of HHV-6. To do this, Dharmacon software (Dharmacon, Lafayette, Colo.) was used to identify potential siRNAs that might target the HHV-6 U51 genes. Out of 24 potential target sites that were identified, 4 siRNAs were found that recognized target sequences which were fully conserved between the two viral variants, HHV-6A and HHV-6B. The selected siRNA targeting sequences were then subjected to a BLAST search against the entire nonredundant nucleotide sequence database in order to ensure that only the intended viral gene would be recognized. These siRNAs were then cloned into a linearized pSuppressorRetro (pSR; Imgenex, San Diego, Calif.) vector downstream of the U6 promoter. To screen the functional activity of these siRNA constructs, HEK293 cells were cotransfected with a plasmid expression vector encoding an SV5 epitope-tagged derivative of HHV-6A U51 plus the various siRNA-carrying pSR vectors (as well as constructs carrying an irrelevant control siRNA). The U51 protein expression level was then examined by Western blot analysis using a monoclonal antibody directed against the SV5 epitope tag. As shown in FIG. 1A, the expression of U51 protein (around 30 kDa) was markedly down-regulated by both si6U51-812 and si6U51-130 (over 80%) but not by the irrelevant siRNA (siNeg. Ctrl.) or the empty vector alone. These results demonstrate that siRNAs can specifically and efficiently inhibit U51 protein expression in mammalian cells in a transient transfection system.

Since the viral envelope glycoprotein B (gB) is known to be essential for replication of herpes simplex-virus type 1, CMV, and other herpesviruses, siRNAs directed against HHV-6 gB were designed for use as a positive control, in experiments aimed at testing the effect of U51-specific siRNAs on viral replication. The gB-specific siRNAs were tested using a similar approach to that described for the U51 siRNAs. As shown in FIG. 1B, transient expression of HHV-6 gB was efficiently blocked (over 90%) by both of the gB siRNAs that were tested. The HHV-6A gB-specific siRNA (si6gBA861) was selected for use in subsequent experiments.

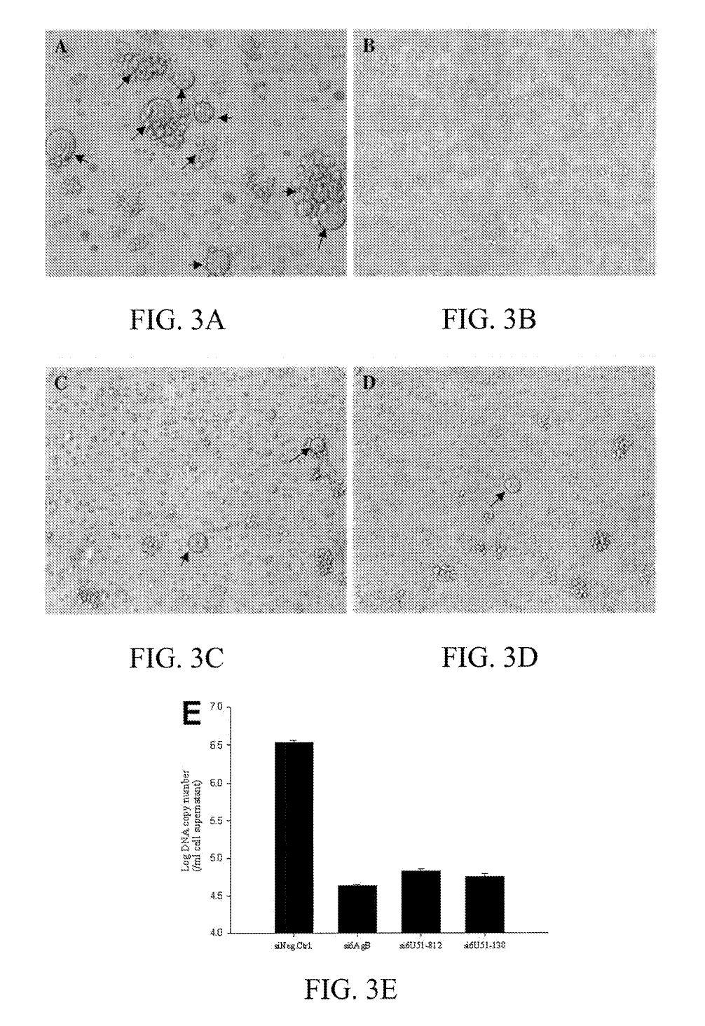

Cell lines stably expressing siRNA-U51 suppressed U51 expression upon virus infection. In order to examine the role of U51 in HHV-6 replication, stable cell lines were derived that expressed one of the U51 siRNAs (si6U51-812 and si6U51-130). For these experiments, the SupT1 cell line was used, which would be highly susceptible to HHV-6A infection. These lymphoid suspension cells are difficult to transfect by standard means (electroporation or lipid-mediated DNA transfer). Retroviral vectors were therefore created that expressed a short hairpin RNA that would be expected to direct the generation of U51-specific short interfering RNA. SupT1 cells were then transduced with recombinant retrovirus particles and subjected to G418-mediated selection, and single colonies were picked and expanded. To confirm the specific gene silencing effect of siRNA-U51 in SupT1 cells, the cells were then infected with HHV-6A, and U51 mRNA levels were quantified 24 h postinfection (FIG. 2). After normalization of U51 expression data (using GAPDH mRNA levels as an internal control), it was determined that U51 mRNA was decreased by over 90% in cells stably expressing si6U51-812 or si6U51-130 relative to unmodified SupT1 cells or SupT1-siNeg.Ctrl. cells that were infected with HHV-6A. Moreover, the growth properties of the clonal, siU51-ex pressing SupT1 sublines were found to be indistinguishable from parental SupT1 cells.

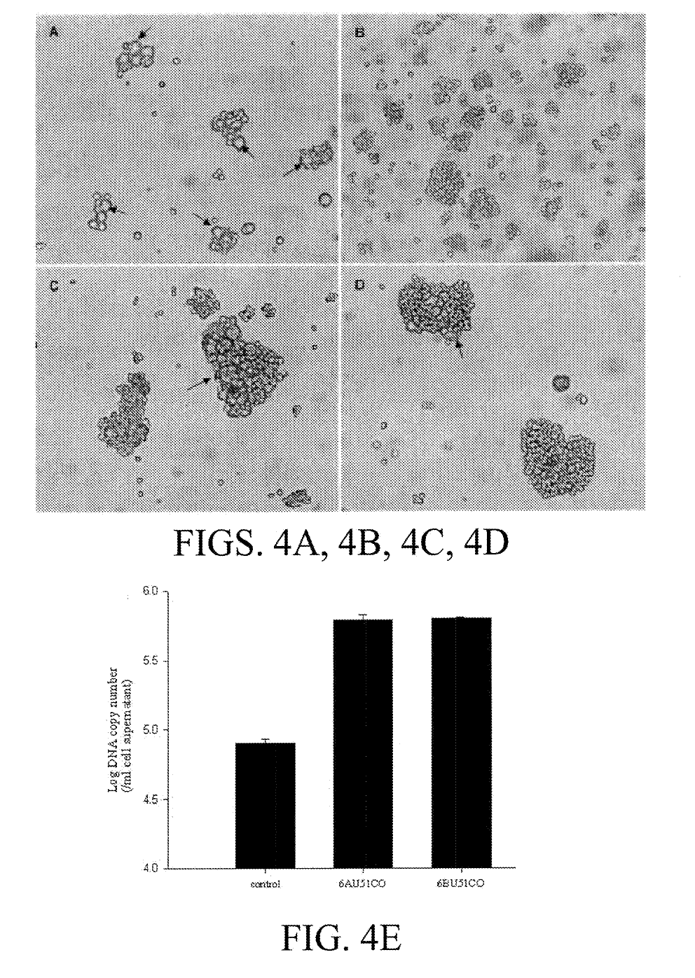

U51-specific siRNA inhibited HHV-6A replication and virally induced syncytium formation. To test whether expression of a U51-specific siRNA would have any effect on virus replication in vitro, a panel of siRNA-expressing SupT1 sublines was infected with HHV-6A strain U1102 at an MOI of 0.1 TCID₅₀/cell. These experiments were performed using several independent clonal SupT1 cell lines, each of which stably expressed a U51-specific siRNA (si6U51-812 or si6U51-130), as well as cells stably expressing si6gB and cells that expressed an irrelevant control siRNA (siNeg.Ctrl). Six days later, when these cultures were examined under the light microscope, a significant reduction was detected in virally induced cytopathic effects (syncytium formation) in those cultures which expressed either the U51-specific siRNA or the gB-specific siRNA; no change in virally induced syncytium formation was detected in cells that expressed the irrelevant control siRNA (FIG. 3A to D).

Virus replication in these cultures was also examined by performing a quantitative real-time DNA PCR assay using TAQMAN® (Roche, Basel, Switzerland) primers and probes specific for the U38 gene (this corresponds to the viral DNA polymerase). As shown in FIG. 3E, virus replication was significantly reduced in the cells that stably expressed either the U51 or the gB-specific siRNA but not in cells that expressed the irrelevant siRNA (siNeg. Ctrl.). Analysis of intracellular viral DNA load was also performed, with very similar results.

To confirm these results, a scrambled derivative of the effective siRNA (si6U51-812) was made and its effect tested on virus replication. Viral replication and syncytium formation were unaltered in cells that expressed this scrambled siRNA, confirming that the result is a sequence-specific effect due to the expressed siRNA.

Expression of a codon-optimized form of U51 can restore virus replication in SupT1 cells that express a U51-specific siRNA. To determine whether the inhibitory effect of the U51 siRNA on virus replication was indeed due to a specific effect on U51 gene expression, an “add-back” experiment was performed. For this purpose, an available, human codon-optimized (CO) version of the U51 ORF was used. This synthetic ORF encodes the authentic U51 protein but does so using altered codons relative to the wild-type U51 gene (Bradel-Tretheway, et al. 2003). As a result, the expression of the codon-optimized U51 ORF should be resistant to inhibition by U51 siRNA. This was verified by performing transient transfection experiments analogous to those shown in FIG. 1A; these studies revealed that the expression of the CO-U51 gene was indeed unaffected by the si6U51-812 siRNA.

A recombinant retrovirus expressing the U51-CO gene was then constructed and used to transduce SupT1 cells that expressed the si6U51-812 siRNA, at an MOI of 10. This construct has previously been shown to result in high levels of U51 expression, both intracellularly and on the surface of all cell types analyzed (Bradel-Tretheway, et al. 2003).

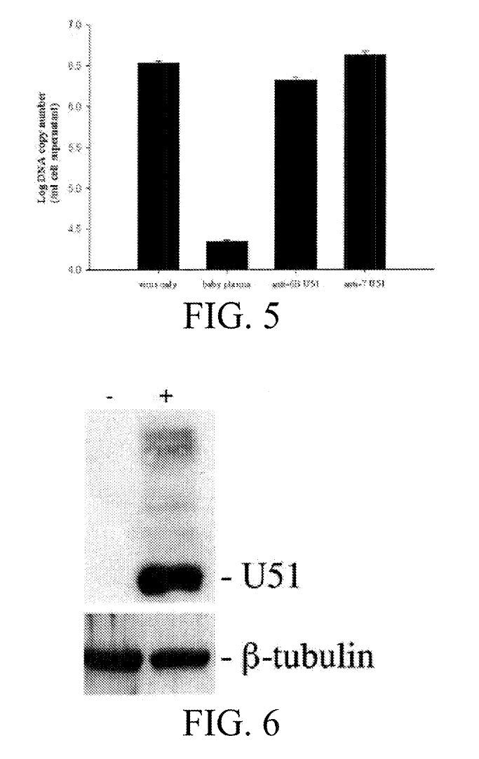

Twenty-four hours after retroviral transduction, the cells were then infected with HHV-6A U1102 at an MOI of 0.1 TCID50/cell. Virally induced cytopathic effects, virus load, and cell growth properties were then measured 6 days later. The results, which are presented in FIG. 4, show that coexpression of the codon-optimized U51 ORF restored virally induced cytopathic effects and viral replication in tire SupT1(si6U51-812) cell line.

Virus infectivity was not affected by a U51-specific antibody. Previous studies have shown that Transmembrane receptors encoded by the human and mouse cytomegaloviruses (UL33, M28) may be incorporated into enveloped virus particles (Margulies, et al. 1996; Oliveira, et al. 2001). This suggested the possibility that HHV-6 U51 might play a role in virion attachment or entry to target cells. This experiment tested the hypothesis.

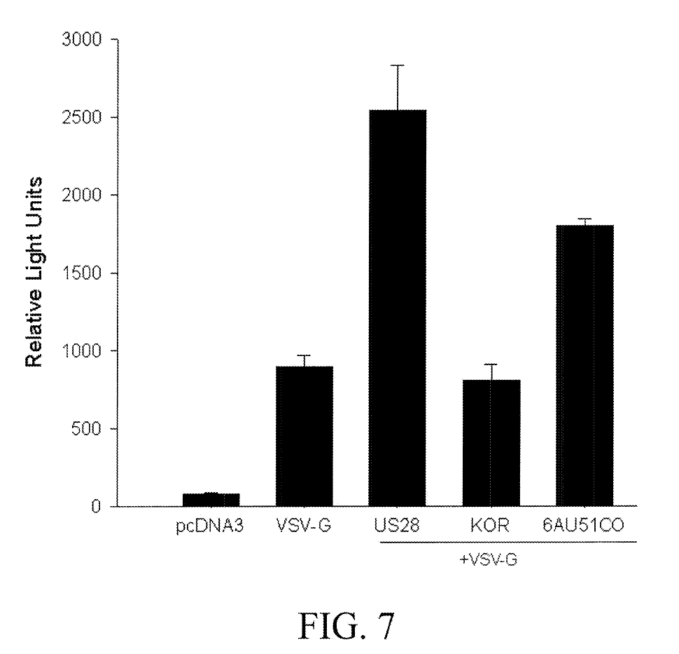

Briefly, HHV-6A virions were mixed with an affinity-purified polyclonal antiserum directed against U51 and then tested whether this had any neutralizing effect on virus infectivity. As controls, an irrelevant antiserum (directed against a nonconserved peptide from HHV-7 U51) was used as well as a human antiserum known to contain high levels of virus-neutralizing antibodies. After incubation with these various antisera for 1 h at 37° C., the HHV-6A inoculum was then added to SupT1 cells, and viral load in culture supernatants was then measured 5 days thereafter by quantitative DNA PCR analysis (FIG. 5). As expected, viral replication was essentially abolished in the cultures that received virions premixed with the positive control human serum. In contrast, there was no significant difference in the level of viral replication in cultures that received untreated virus inocula, inocula preincubated with the HHV-6 U51-specific antiserum, or inocula that were treated with the irrelevant antiserum. It is important to note that the U51-specific antiserum was not heat inactivated and thus would have been expected to be capable of mediating complement directed lysis of virus particles had it bound to cell-free virions. Thus, these data suggest that U51 is most likely not involved in the initial interaction between HHV-6 virions and their target cells. However, this does not rule out the possibility that U51 may be involved either in modulating host cell signaling, so as to favor more efficient virus replication, or in the cell-cell spread of virus, perhaps by promoting fusion of virus-infected cells with virus-negative targets.

U51-mediated cell signaling. In order to examine whether U51 might contribute to cell signaling events, a series of experiments were performed to examine both ligand binding and G protein coupling. For this set of experiments, particular attention was paid to the possibility that U51 might interact with opioid ligands in light of the previously noted similarity between U51 and human opioid receptors (Gompels, et al. 1995). For initial ligand binding experiments, cells were transfected with recombinant adenovirus vectors that encoded a human codon-optimized form of U51, because it has been shown that codon optimization will enhance U51 expression 10- to 100-fold in mammalian cells (Bradel-Tretheway, et al. 2003). As noted previously, use of the codon-optimized constructs permits cell surface expression of U51, even in cell lines that are not of T-cell lineage (Bradel-Tretheway, et al. 2003); this contrasts with results reported by Menotti and colleagues, using a non-codon-optimized expression system that probably resulted in lower total levels of protein expression (Menotti, et al. 1999).

Briefly, the ligand binding studies revealed that membranes from cells which expressed tire HHV-6B U51 protein did not specifically bind the μ-selective opioid, [³H]DAMGO, the δ-selective opioid, [³H]naltrindole or [³H]DPDPE, or the κ agonist, [³H]U69,593 or [³H]bremazocine. Also, HHV-6B U51 did not specifically bind the nonselective opioid receptor antagonist [³H]diprenorphine.

The [³⁵S]GTPγS assay was then used to determine if opioids or a selected subset of chemokines could stimulate [³⁵S]GTPγS binding mediated by HHV-6B U51. Three different sets of HEK293 cell membranes were used in experiments, including those from wild-type 293 cells and cells stably transfected with a Tet-inducible expression plasmid carrying HHV-6B U51 (membranes were prepared from these cells either in the absence of U51 induction or following addition of 1 μg/ml tetracycline for 24 h, which resulted in a 50- to 100-fold induction of U51 expression at both the RNA and protein levels [FIG. 6]). Membranes from these different sets of HEK293 cells were tested with chemokines and opioids to determine if any chemokines or opioids stimulated the coupling of the U51 protein to G proteins. Table 1 shows that none of the chemokines or opioids tested had a significant effect on [³⁵S]GTPγS binding. Overall, no evidence was found for opioid ligand binding or opioid-induced G protein coupling by HHV-6B U51, and attention was therefore turned to the possibility that U51 influences cell membrane fusion events, as has been described previously for HCMV US28 (Pleskoff, et al. 1998).

TABLE 1
Effect of chemokines and opioids on [³⁵S]GTPγS binding

| | % Control [35S]GTPγS binding ± SEM | | | |
|---|---|---|---|---|
| | Control | | U51 | U51 |
| Compound | Cell Eₘₐₓ^{a} | Native | (Unind) | (+Tet) |
| ICI 1 μM | | 104 ± 5 | 103 ± 5 | 97 ± 2 |
| RANTES, 100 ng/ml | | 101 ± 2 | 111 ± 1 | 104 ± 2 |
| MCP-3, 1 ng/ml | | 97 ± 3 | 109 ± 9 | 103 ± 2 |
| Lymphotactin, 100 ng/ml | | 97 ± 2 | 99 ± 3 | 104 ± 7 |
| Interleukin-8, 100 ng/ml | | 101 ± 5 | 96 ± 2 | 96 ± 3 |
| Morphine, 1 μM | | 99 ± 9 | 99 ± 0.9 | 91 ± 9 |
| U50, 488, 1 μM | 177 ± 10 | 97 ± 5 | 100 ± 3 | 90 ± 8 |
| Deltorphin II, 1 μM | 234 ± 15 | 100 ± 4 | 98 ± 2 | 91 ± 6 |
| DPDPE, 1 μM | 254 ± 16 | 104 ± 1 | 96 ± 1 | 99 ± 2 |
| Naltrindole, 1 vM | | 96 ± 4 | 98 ± 4 | 97 ± 4 |
| DAMGO, 1 μM | 216 ± 4.4 | 106 ± 4 | 96 ± 3 | 86 ± 6 |

^{a}Three different sets of HEK 293 cell membranes were used in experiments, including those from wild-type 293 cells and cells stably transfected with a Tet-regulatable HHV-6B U51 expression plasmid; membranes from the latter cells were prepared after culturing cells either in the absence of tetracycline (uninduced; Unind) or following induction with 1 μg/ml tetracyline for 24 h (which resulted in a reproducible 50- to 100-fold upregulation of U51 expression at both the RNA and protein level; FIG. 6). [³⁵S]GTPγS binding to these membranes was performed as described in Materials and Methods. [³⁵S]GTPγS binding to the HEK membranes in the absence of added compound was set as 100% control binding, Date are the mean percent control binding standard errors of the means from three experiments performed in triplicate. The control cells were CHO cells that had been stably transfected with the human μ, δ, or κ opioid receptor. [³⁵S]GTPγS binding to these cell membranes was measured as previously described (Parkhill, et al. 2002). The values reported are the Eₘₐₓ values obtained for the selective opioids. Eₘₐₓ values were obtained at an opioid concentration of less that 1 μM.

Coexpression of U51 and vesicular stomatitis virus (VSV) G glycoprotein enhanced cell fusion. Membrane fusion events are important for viral entry into host cells and also for cell-to-cell spread of virus. To examine whether U51 facilitates virus replication and spread by contributing to membrane fusion, a luciferase-based gene reporter assay was used to quantitate cell fusion events. This assay relies on the presence of the HIV-1 transactivating protein (Tat) in one cell and a Tatinducible reporter gene cassette (firefly luciferase linked to the HIV-1 LTR) in the other cell. Upon fusion of the target and effector cells, Tat will activate luciferase transcription, and luciferase expression can men be detected and quantitated by a luminometer. Because the contents of tire effector and target cells must mix in order for the HIV Tat to transcribe the luciferase gene, the level of luciferase activity represents the extent of fusion between foe effector and target cells.

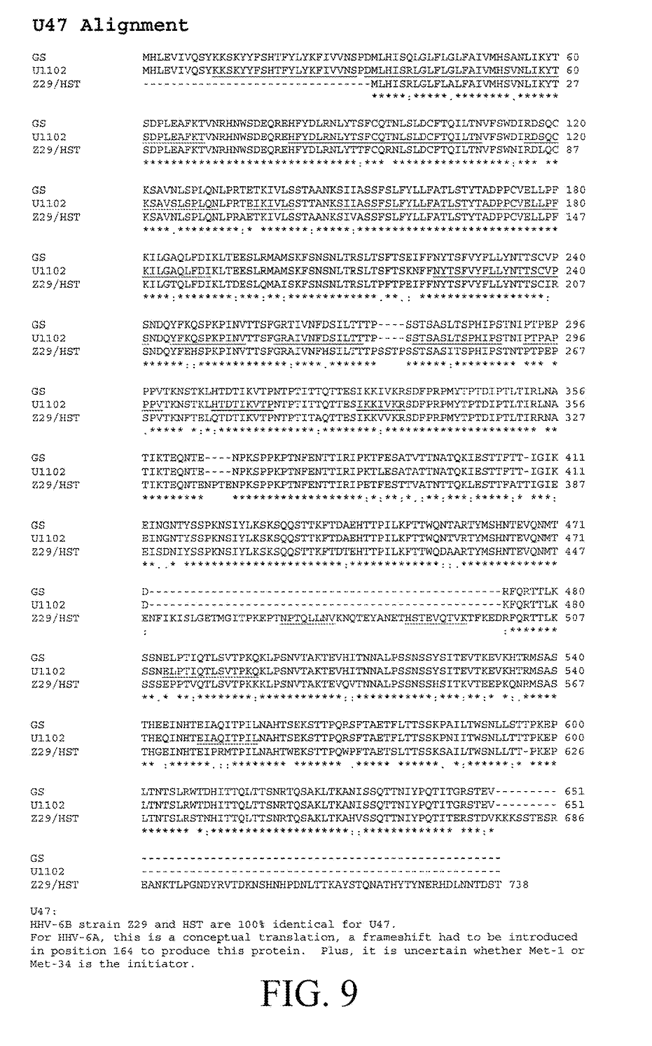

Equal numbers of HEK293 cells were transiently transfected with a vector expressing either HIV Tat or luciferase under the transcriptional control of the HIV LTR. All cells also received a plasmid clone encoding pVSV-G, in foe presence or absence of expression vectors that carry HHV-6 U51, the rat kappa opioid receptor (as a negative control), or HCMV US28 (as a positive control) (Pleskoff, et al. 1998). Four hours after transfection, the two cell populations were trypsinized and mixed together at a 1:1 ratio. Forty-four hours thereafter, the cell fusion activity was quantitatively determined by measuring luciferase gene expression in the lysates of the cocultured cells (FIG. 7). Cells coexpressing US28 and VSV-G exhibited an increased level of fusion activity (˜3-fold) compared to cells transfected with VSV-G alone. Cells coexpressing VSV-G plus HHV-6A U51 also showed enhanced high fusion activity (˜2-fold) compared to cells transfected with VSV-G alone, while the kappa opioid receptor expression plasmid had no effect on cell fusion.

### Example 2

### Peptide ELISA Test for Detection of Human Herpesvirus (BHV)-6A Specific Antibodies

Provided are peptide sequences that can be used to develop a peptide ELISA test, for the detection of human herpesvirus (HHV)-6A specific antibodies. These peptides can be used alone, or in various combinations, to develop the variant-specific ELISA. Previously defined antibody epitopes, which are known to differ in HHV-6A versus HHV-6B, can be used herein. One or more these epitopes should be differentially recognized by sera from persons infected with HHV-6A versus persons infected with HHV-6B alone. Known HHV-6 epitopes are listed in Table 2.

TABLE 2

Previously defined linear antibody
epitopes, which differ in HHV-6A and HHV-6B

| | | | |
|---|---|---|---|
| Sequence | SEQ ID | Gene | Comment |

| | | | |
|---|---|---|---|
| EKILEVSN (6A) | SEQ ID NO:23 | 101K | C3108-101 is 6B |
| ERILEVSD (6B) | SEQ ID NO:24 | [U11] | specific; Asp723 is key |
| | | | (Pellett, et al. 1993) |
| KYYDKNIYF (A-GS) | SEQ ID NO:25 | gQ | 2D6 is a neutralizing |
| KYYDDSIYF (B) | SEQ ID NO:26 | (gp105) | Mab; reacts to HHV6A |
| | | [U100] | (Pfeiffer, et al 1993) |
| NVTISRYRW (A) | SEQ ID NO:27 | gH [U48] | OHV3 MoAb site; reacts |
| NVTISKYKW (B) | SEQ ID NO:23 | | to HHV6B; the Arg is |
| | | | key (Takeda, et al. 1997) |
| DDGKGDRSHKNEDESALASK (A) | SEQ ID NO:29 | P41/38 | S328 is key for C5 |
| DDGKGDRNHKNEDESALVSK (A) | SEQ ID NO:30 | [U27] | MoAb reactivity (A- |
| | | | specific) (Xu, Y, et al. |
| | | | 2001) |

Variant-specific epitopes have also been mapped to a conformational region with gB (U39; amino acids 335-395; Takeda, et al. 1996) and to an unknown region of IE2 (U86-91; Arsenault, et al. 2003). Peptide mimetics of these conformational epitopes can be identified by selective screening of the relevant monoclonal antibodies against random peptide libraries (e.g., phage display libraries).

Also provided are unique segments/regions within HHV-6A encoded proteins that diverge from the corresponding sequence in HHV-6B. Sequences with high potential for utility in an ELISA assay were selected on the basis of this and two additional criteria—(1) that the proteins from which the peptides are derived are expected to be either highly abundant, immunodominant or both, and (2) that the peptide domains have high potential for surface display and antibody recognition/immunogenicity.

Peptides were selected by performing an alignment of the predicted amino acid sequences for specific genes of interest in the HHV-6A and HHV-6B genomes. Genes selected for this analysis included abundant and immunodominant tegument proteins (U11, U14), abundant and highly variable immediate-early transactivators (U86, U90, U95), and highly expressed virion glycoproteins (U47, U54).

Peptides that showed significant variation between HHV-6A and HHV-6B were then subjected to further analysis, to identify predicted antigenic sequences (Molecular Immunology Foundation). Peptides meeting criteria for (i) divergence in HHV-6A and HHV-6B, (ii) predicted antigenicity and (iii) derivation from proteins expected to be major immune targets are listed below (Table 3). A more complete listing is found in Table 4.

TABLE 3

Predicted linear antibody epitopes,
which differ in HHV-6A and HHV-6B

Sequences

| | | |
|---|---|---|
| Gene | HHV6A (U1102) | HHV6B (Z29) |

| | | | | |
|---|---|---|---|---|
| U11 | NLDLPLSS | (SEQ ID NO:31) | SGVEPLSS | (SEQ ID NO:32) |
| | EKILEVSN | (SEQ ID NO:23) | ERILEVSD | (SEQ ID NO:24) |
| | KKSKYYFSHTFYLYKFIVVNS | (SEQ ID NO:35) | (Absent) | |
| U47 | (Absent) | | NPTQLLNV | (SEQ ID NO:36) |
| | (Absent) | | HSTECQTVK | (SEQ ID NO:37) |
| U14 | DLFEKVLRLGV | (SEQ ID NO:38) | DLLMSVTFRLGV | (SEQ ID NO:39) |
| | NIIPNTVTPVH | (SEQ ID NO:40) | NIVSNLITPFH | (SEQ ID NO:41) |
| | SQPIOVVYFFP | (SEQ ID NO:42) | SQELOKYFFFP | (SEQ ID NO:43) |
| U54 | FTNQAVLRTPSLSTVANL | (SEQ ID NO:44) | LASQPVSRAPSLTTVAHV | (SEQ ID NO:45) |
| | ADLPYEHYTYP | (SEQ ID NO:46) | ADLSYQQYMHP | (SEQ ID NO:47) |
| | ENQVLTPDVIS | (SEQ ID NO:48) | KQQVSTSPDAIS | (SEQ ID NO:49) |
| | QNFKEVSVKN | (SEQ ID NO:50) | QDVREVAVKN | (SEQ ID NO:51) |
| | VKTIIQSPSPYCKLKNPSIMDKN | (SEQ ID NO:52) | NETIIPSTSACPTQETPSTMNRN | (SEQ ID NO:53) |
| U86 | DSETVVRR | (SEQ ID NO:54) | DSLNTPKR | (SEQ ID NO:55) |
| | PNITTSHLQGKQNVRLHN | (SEQ ID NO:56) | PKTTNITTNTIYRPRDNQSNISRN | (SEQ ID NO:57) |
| | EPDDLCYRDYVRLKERKVS | (SEQ ID NO:58) | EIEEELSYREYVRRKEKKES | (SEQ ID NO:59) |
| | (Absent) | | NIIQPFSQLF | (SEQ ID NO.60) |
| | LPKAADVIV | (SEQ ID NO:61) | LPETTNVIV | (SEQ ID NO:62) |
| | TSEHKQLHL | (SEQ ID NO:63) | TSEDNYLHL | (SEQ ID NO:64) |
| U90 | TATQVFDPPVT | (SEQ ID NO:65) | AETQIFDPQGT | (SEQ ID NO:66) |
| | (Absent) | | QQDILAYSP | (SEQ ID NO:67) |
| | (Absent) | | SATPVKSH | (SEQ ID NO:68) |
| U95 | KYYDKNIYF | (SEQ ID NO:25) | KYYDDSIYF | (SEQ ID NO:26) |

Underlined sequences particularly promising domains (especially variable). Hence. the HHV-6A peptides based on these sequences should NOT react with antibodies specific to HHV-6B

Screening of the blood supply, to prevent potential spread of HHV-6A to uninfected individuals. HHV-6A is believed to be rare in the general population (on the order of perhaps 1% of US adults) and has been associated with serious disease in immunocompromised individuals (Singh, et al 2000; Zerr, et al. 2001), as well as with multiple sclerosis Akhyani, et al. 2000; Cermelli, et al. 2003; Challoner, et al. 1995; Friedman, et al. 2005; Goodman, et al. 2003; Opsahl, et al. 2005; Tejada-Simon, et al. 2003) and chronic fatigue syndrome (Josephs, et al. 1991; Komaroff, 1988).

Advances in donor screening and blood testing have dramatically improved blood safety. All blood donated at American Red Cross blood centers nationwide, approximately 50 percent of the nation's blood supply, is currently screened using the following tests to reduce the risk of disease transmission: HIV/AIDS (HIV-I Antibody test, HIV-1/2 Antibody test; HIV-I p24 Antigen test); Hepatitis B (Hepatitis B Surface Antigen; Hepatitis B Core Antibody); Hepatitis C (Anti-HCV); Hepatitis (ALT); Syphilis (Serologic test for syphilis—TP or RPR); Human T-cell Lymphotropic Virus (HTLV) (HTLV-I Antibody test; HTLV-I/II Antibody test); Hepatitis C and HIV/AIDS (Nucleic Acid Testing (NAT)); West Nile Virus (Nucleic Acid Testing (WNV-NAT)). CMV testing is also performed on some units of blood for patients who require CMV negative blood, for example, neonates weighing less than 1500 grams, and immuno-compromised or immune-suppressed patients.

Seroepidemiohgic studies on HHV-6A. These studies were performed to identify the distribution of HHV-6A infection and to better understand the relationship between HHV-6A infection and human disease. Prior to the present invention, HHV-6A could only be detected by a DNA-based PCR test for the presence of virus DNA in blood or other body fluids/tissues. This PCR test is insensitive at best.

TABLE 4

Divergent peptides with predicted
antigenicity, for selected HHV-6A genes.

| | |
|---|---|
| Gene | Sequence |

| | | |
|---|---|---|
| | QRHPIPFA | SEQ ID NO:33 |
| | YNLLVLWLMYHYVLS | SEQ ID NO:34 |
| | LMDFVPLRG | SEQ ID NO:69 |
| | IHSNLTLPS | SEQ ID NO:70 |
| | QPKFLELDS | SEQ ID NO:71 |
| | EKILLKE | SEQ ID NO:72 |
| | NLDLPLSS | SEQ ID NO:31 |
| U11 | IGPSGILDFNVKFPPN | SEQ ID NO:73 |
| | FLDPVHRFVPE | SEQ ID NO:74 |
| | SPRNVFLIK | SEQ ID NO:75 |
| | VNNLLSQFTNLIS | SEQ ID NO:76 |
| | EKILEVSN | SEQ ID NO:23 |
| | IDLALEKVKV | SEQ ID NO:77 |
| | QDESFVPAQLMK | SEQ ID NO:78 |
| | SGPGVAESLD | SEQ ID NO:79 |
| | KKSKYYFSHTFYLYKFIVVNS (*) | SEQ ID NO:35 |
| | DMLHISRLGLFLGLFAIVMHSVNLIKYT | SEQ ID NO:80 |
| | HFYDLRNLYTSFCQTNLSLDCFTQILTN | SEQ ID NO:81 |
| | RDSQCKSAVSLSPLQN | SEQ ID NO:82 |
| | EIKIVLS | SEQ ID NO:83 |
| U47 | YFKQSPKPINV | SEQ ID NO:84 |
| | GRAIVNFDSILTT | SEQ ID NO:85 |
| | PTPAPPPV | SEQ ID NO:86 |
| | ELPTIQTLSVTPKQ | SEQ ID NO:87 |
| | EIAQITPIL | SEQ ID NO:88 |
| | NPTQLLNV | SEQ ID NO:36 |
| | (HHV6B; absent in HHV6A) | |
| | HSTECQTVK | SEQ ID NO:37 |
| | (HHV6B; absent in HHV6A) | |
| | YIKDVLIQ | SEQ ID NO:89 |
| | EQIMVIITK | SEQ ID NO:90 |
| | LFEKVLRLGVHIN | SEQ ID NO:91 |
| | FTKAVKLIN | SEQ ID NO:92 |
| | LYKIPHYTLKEAVDVYS | SEQ ID NO:93 |
| U14 | IYNCKVQI | SEQ ID NO:94 |
| | NSIVEDCVLVGFQLPD | SEQ ID NO:95 |
| | KDLFSHYKLILEKLFEISIF | SEQ ID NO:96 |
| | ILPTFIKSHLIEF | SEQ ID NO:97 |
| | KLPIQVDP | SEQ ID NO:98 |
| | SYDKIIDVEENVIQVL | SEQ ID NO:99 |
| | AVANKYGLSLPQVIK | SEQ ID NO:100 |
| U39 | MIYCDPDHYIRA | SEQ ID NO:101 |
| | EANLVNSHAQCYSAVAM | SEQ ID NO:102 |
| | PGELRLFKCGLITPPSSAVVCICRD | SEQ ID NO:103 |
| | FTSSPFTY | SEQ ID NO:104 |
| | NISNLPVQR | SEQ ID NO:105 |
| | AHDRHIIPHGQCDAKFVIYGPLTRIKIQVAD | SEQ ID NO:106 |
| | AEIWVNLQN | SEQ ID NO:107 |
| | SHKNVYISRILL | SEQ ID NO:108 |
| | YPEQLAIQISLTP | SEQ ID NO:109 |
| | TLLKCNTHSITVCATK | SEQ ID NO:110 |
| U54 | IPNTVTPVHCSF | SEQ ID NO:111 |
| | FTGLFIPKLLLGI | SEQ ID NO:112 |
| | NYSQPIGVVYFFPKQIL | SEQ ID NO:113 |
| | ASKIYVN | SEQ ID NO:114 |
| | FTNQAVLRTPSLSTVANL | SEQ ID NO:115 |
| | IFLSSLRVAF | SEQ ID NO:116 |
| | TAHMVPLHFSL | SEQ ID NO:117 |
| | QNLTILEGDVGIHFI | SEQ ID NO:118 |
| | LEESLMCDT | SEQ ID NO:119 |
| | FDDLIIPGLESFGLIIP | SEQ ID NO:120 |
| | DVIQSAMKLSGLYCDA | SEQ ID NO:121 |
| U86 | QDPIYSQE | SEQ ID NO:122 |
| | QDPRIVAQTHRQCTSSAS | SEQ ID NO:123 |
| | GSTQVRFASELPNQLLQPM | SEQ ID NO:124 |
| | TSLPYQPYR | SEQ ID NO:125 |
| | YNFRHHPY | SEQ ID NO:126 |
| | SKYQQPYKRCFT | SEQ ID NO:127 |
| | RSYDCSD | SEQ ID NO:128 |
| | SADLPYEHYTY | SEQ ID NO:129 |
| | DSTHVQS | SEQ ID NO:130 |
| | ENQVLTPDVISLSYRP | SEQ ID NO:131 |
| | VDIQKYKKAHIRCRSVQ | SEQ ID NO:132 |
| | SKLNPLLSPLPLTPEPAIDF | SEQ ID NO:133 |
| | LQDTVPISKHTP | SEQ ID NO:134 |
| | NFKEVSVKNV | SEQ ID NO:135 |
| | KSKTHHYS | SEQ ID NO:136 |
| | YKSPVKTIIQSPSPYCKLKN | SEQ ID NO:137 |
| | KHLSKSCTM | SEQ ID NO:138 |
| | ELRQIYCD | SEQ ID NO:139 |
| | SMSRCKSHCRN | SEQ ID NO:140 |
| | DSLTVVRR | SEQ ID NO:141 |
| | RSNSHIVTG | SEQ ID NO:142 |
| | FTPFYYQ | SEQ ID NO:143 |
| | SSSSSSASLSCSK | SEQ ID NO:144 |
| | TLKTCRK | SEQ ID NO:145 |
| | TTSHLQG | SEQ ID NO:146 |
| | EPDDLCYRDYVRLKE | SEQ ID NO:147 |
| | LKEAVYDICCN | SEQ ID NO:148 |
| | RSKKVAQIIK | SEQ ID NO:149 |
| | KRFIQLQK | SEQ ID NO:150 |
| | HDLFSRHSDVKTMIIYAATPIDFVGAVKTCNK | SEQ ID NO:151 |
| | MFIGLGLEQLSQLININLLSSASTKYVESYSK | SEQ ID NO:152 |
| | SRNLLLD | SEQ ID NO:153 |
| | IIKAVKDIFSKATV | SEQ ID NO:154 |
| | TLDCQK | SEQ ID NO:155 |
| | SKDFCEK | SEQ ID NO:156 |
| | CDKAFLKLNVNCKNLITAA | SEQ ID NO:157 |
| | ANTILQSIVICSN | SEQ ID NO:158 |
| | SWQHLKLLRR | SEQ ID NO:159 |
| | ITQACECLE | SEQ ID NO:160 |
| | GLIKPLTPLQIM | SEQ ID NO:161 |
| | KMYPCTPSPEVPGKSKYVG | SEQ ID NO:162 |
| | NPNCVGTASVTD | SEQ ID NO:163 |
| U90 | SISGLQSCKN | SEQ ID NO:164 |
| | LLERLLDTQCDSVVE | SEQ ID NO:165 |
| | FSNSICSPPEVTPSKK | SEQ ID NO:166 |
| | AKRKHVSS | SEQ ID NO:167 |
| | QLPKAADVIVI | SEQ ID NO:168 |
| | GNSILIKA | SEQ ID NO:169 |
| | TSEHKQLHLSDY | SEQ ID NO:170 |
| | GHCPSYGFPTPVFTI | SEQ ID NO:171 |
| | QVDNCPI | SEQ ID NO:172 |
| | EAKHCFMNHFVPI | SEQ ID NO:173 |
| | IPTKKLIID | SEQ ID NO:174 |
| | ITKHCQDLCNKYNVVTP | SEQ ID NO:175 |
| | TATQVFDP | SEQ ID NO:176 |

* Possibly absent in HHV6B; it depends on which Met-start codon is used.
Listed epitopes have 2 or more differences between HHV6A and HHV6B.
Listed peptides correspond to HHV6A sequence, unless otherwise indicated.

All references cited herein are each incorporated by reference in their entirety.

### REFERENCES

Ablashi, D. V. et al., 1993. Human herpesvirus-6 strain groups: a nomenclature. Arch Virol 129:363-6.
Ablashi, D. V. et al., 1991. Genomic polymorphism, growth properties, and immunologic variations in human herpesvirus-6 isolates. Virology 184:545-52.
Ahlqvist, I. et al., 2005. Differential tropism of human herpesvirus 6 (HHV-6) variants and induction of latency by HHV-6A in oligodendrocytes. J Neurovirol 11:384-94.
Akashi, K. et al., 1993. Brief report: severe infectious mononucleosis-like syndrome and primary human herpesvirus 6 infection in an adult. N Engl J Med 329:168-71.
Akhyani, N. et al., 2000. Tissue distribution and variant characterization of human herpesvirus (HHV)-6: increased prevalence of HBV-6A in patients with multiple sclerosis. J Infect Dis 182:1321-5.
Alkhatib, G. et al., 1996. CC CKR5: a RANTES, MIP-1alpha, MIP-1beta receptor as a fusion cofactor for macrophage-tropic HIV-1. Science 272:1955-1958.
Arsenault, S. et al., 2003. Generation and characterization of a monoclonal antibody specific for human herpesvirus 6 variant A immediate-early 2 protein. J Clin Virol 28:284-90.
Arvanitakis, L. et al., 1997. Human herpesvirus KSHV encodes a constitutively active G-protein-coupled receptor linked to cell proliferation. Nature 385:347-350.
Aubin, J. T. et al., 1991. Several groups among human herpesvirus 6 strains can be distinguished by Southern blotting and polymerase chain reaction. J. Clin. Microbiol. 29:367-372.
Beisser, P. S. et al., 1998. The R33 G protein-coupled receptor gene of rat cytomegalovirus plays an essential role in the pathogenesis of viral infection. J. Virol. 72:2352-2363.
Beisser, P. S. et al., 1999. Deletion of the R78 G protein-coupled receptor gene from rat cytomegalovirus results in an attenuated, syncytium-inducing mutant strain. J. Virol. 73:7218-7230.
Bhuyan, P. K. et al., 2004. Short interfering RNA-mediated inhibition of herpes simplex virus type 1 gene expression and function during infection of human keratinocytes. J. Virol. 78:10276-10281.
Billstrom, M. A. et al., 1998. Intracellular signaling by the chemokine receptor US28 during human cytomegalovirus infection. J. Virol. 72:5535-5544,
Bitsch, A., and W. Brack. 2002. Differentiation of multiple sclerosis subtypes: implications for treatment, CNS Drugs 16:405-18.
Bradel-Tretheway, B. G., Z. Zhen, and S. Dewhurst 2003. Effects of codonoptimization on protein expression by the human herpesvirus 6 and 7 U51 open reading frame. J. Virol. Methods 111:145-156.
Bradford, M. M. 1976. A rapid and sensitive method for the quantitation of microgram quantities of protein utilizing the principle of protein-dye binding. Anal. Biochem. 72:248-254,
Capra, V. et al., 2004. Mutational analysis of the highly conserved ERY motif of the thromboxane A2 receptor: alternative role in G protein-coupled receptor signaling. Mol Pharmacol 66:880-9.
Carrigan, D. R., D. Harrington, and K. K. Knox. 1996. Variant A human herpesvirus six as a cofactor in the pathogenesis of AIDS. J Acquir Immune Defic Syndr Hum Retrovirol 13:97-8.
Casarosa, P. et al., 2001. Constitutive signaling of the human cytomegalovirusencoded chemokine receptor US28. J. Biol Chem, 276:1133-1137.
Casarosa, P. et al., 2003. Constitutive signaling of the human cytomegalovirusencoded receptor UL33 differs from that of its rat cytomegalovirus homolog R33 by promiscuous activation of G proteins of the Gq, Gi, and Gs classes. J. Biol. Chem. 278:50010-50023.
Caserta, M. T. et al., 1994. Neuroinvasion and persistence of human herpesvirus 6 in children. J Infect Dis 170:1586-9.
Caserta, M. T. et al., 2004. Human herpesvirus 6 (HHV6) DNA persistence and reactivation in healthy children. J Pediatr 145:478-84,
Cermelli, C. et al., 2003. High frequency of human herpesvirus 6 DNA in multiple sclerosis plaques isolated by laser microdissection. J Infect Dis 187:1377-87.
Challoner, P. B. et al. 1995. Plaque-associated expression of human herpesvirus 6 in multiple sclerosis. Proc Natl Acad Sci USA 92:7440-4,
Chan. P. K. et al., 1999. Presence of human herpesviruses 6, 7, and 8 DNA sequences in normal brain tissue. J Med Virol 59:491-5.
Clark, D. A., V. C. Emery, and P. D. Griffiths. 2003. Cytomegalovirus, human herpesvirus-6, and human herpesvirus-7 in hematological patients. Semin Hematol 40:154-62.
Cone, R. W., M. L. Huang, R, C. Hackman, and L. Corey. 1996. Coinfection with human herpesvirus 6 variants A and B in lung tissue. J Clin Microbiol 34:877-81.
Davis T. L, et al., Structural and molecular characterization of a preferred protein interaction surface on G protein beta gamma subunits. Biochemistry. 2005 Aug. 9; 44(31): 10593-604
Davis-Poynter, N. J. et al., 1997. Identification and characterization of a G protein-coupled receptor homolog encoded by murine cytomegalovirus. J. Virol. 71:1521-1529,
De Bolle, L. et al., 2005. Quantitative analysis of human herpesvirus 6 cell tropism. J Med Virol 75:76-85.
De Clercq, E, 2003. Clinical potential of the acyclic nucleoside phosphonates eidofovir, adefovir, and tenofovir in treatment of DNA virus and retrovirus infections. Clin Microbiol Rev 16:569-96.
Dewhurst, S, 2004. Human herpesvirus type 6 and human herpesvirus type 7 infections of the central nervous system. Herpes 11 Suppl 2:105A-111A.
Dewhurst, S. et al., 1992. Phenotypic and genetic polymorphisms among human herpesvirus-6 isolates from North American infants. Virology 190:490-3.
Dewhurst, S. et al., 1993. Human herpesvirus 6 (HHV-6) variant B accounts for the majority of symptomatic prim airy HHV-6 infections in a population of U.S. infants, J Clin Microbiol 31:416-8.
Dhanak, D. et al., 2000. Metal mediated protease inhibition: design and synthesis of inhibitors of the human cytomegalovirus (hCMV) protease. Bioorg Med Chem Lett 10:2279-82.
Dhanak, D. et al., 1998. Benzothiopyran-4-one based reversible inhibitors of the human cytomegalovirus (HCMV) protease. Bioorg Med Chem Lett 8:3677-82.
Dollard, S. C. et al., 1994. Enhanced responsiveness to nuclear factor kappa B contributes to the unique phenotype of simian immunodeficiency virus variant SIVsmmPBj14. J. Virol. 68:7800-7809.
Dominguez, G. et al., 1999. Human herpesvirus 6B genome sequence: coding content and comparison with human herpesvirus 6A. J. Virol 73:8040-8052.
Donati, D. et al., 2005. Variant-specific tropism of human herpesvirus 6 in human astrocytes. J Virol 79:9439-48.
Donati, D. et al., 2003. Detection of human herpesvirus-6 in mesial temporal lobe epilepsy surgical brain resections. Neurology 61:1405-11.
Doranz, B. J. et al., 1996, A dual-tropic primary HIV-1 isolate that uses fusin and the beta-chemokine receptors CKR-5, CKR-3, and CKR-2b as fission cofactors. Cell 85:1149-1158.
Feng, Y. et al., 1996. HIV-1 entry cofactor; functional cDNA cloning of a seven-transmembrane, G protein coupled receptor. Science 272:872-877,
Fitzsimons C. P. et al., Chemokine-directed trafficking of receptor stimulus to different Gproteins: selective inducible and constitutive signaling by human herpesvirus 6-encoded chemokine receptor U51. Mol Pharmacol 2005
Friedman, J. E. et al., 2005. A randomized clinical trial of valacyclovir in multiple sclerosis. Mult Scler 11:286-95.
Gao, J. L. and P. M. Murphy. 1994. Human cytomegalovirus open reading frame US28 encodes a functional beta chemokine receptor. J. Biol. Chem. 269:28539-28542.
Gershengorn, M. C. et al., 1998. Chemokines activate Kaposi's sarcoma-associated herpesvirus G protein coupled receptor in mammalian cells in culture. J. Clin. Investig. 102:1469-1472.
Gompels, U. A. et al., 1995. The DNA sequence of human herpesvirus-6: structure, coding content, and genome evolution. Virology 209:29-51.
Goodman, A. D. et al., 2003. Human herpesvirus 6 genome and antigen in acute multiple sclerosis lesions. J Infect Dis 187:1365-76.
Gordon, L. et al., 1996. Detection of herpes simplex virus (types 1 and 2) and human herpesvirus 6 DNA in human brain tissue by polymerase chain reaction. Clin Diagn Virol 6:33-40.
Grivel, X. C. et al., 2003. Pathogenic effects of human herpesvirus 6 in human lymphoid tissue ex vivo. J Virol 77:8280-9.
Hall, C. B. et ah, 1994. Human herpesvirus-6 infection in children. A prospective study of complications and reactivation. N Engl J Med 331:432-8.
Hall, C. B. et al., 1998. Persistence of human herpesvirus 6 according to site and variant: possible greater neurotropism of variant A. Clin Infect Dis 26:132-7.
Holy, A. et al., 2002. 6-[2-(Phosphonomedioxy)alkoxy]pyrimidines with antiviral activity. J Med Chem 45:1918-29.
Hoog, S. S. et al., 1997. Active site cavity of herpesvirus proteases revealed by the crystal structure of herpes simplex virus protease/inhibitor complex. Biochemistry 36:14023-9.
Hulshof, J. W. et al., 2005. Synthesis and structure-activity relationship of the first nonpeptidergic inverse agonists for the human cytomegalovirus encoded chemokine receptor US28. J Med Chem 48:6461-71.
Isegawa, Y. et al., 1999. Comparison of the complete DNA sequences of human herpesvirus 6 variants A and B. J. Virol. 73:8053-8063.
Isegawa, Y. et al., 1998. Human herpesvirus 6 open reading frame U12 encodes a functional betachemokine receptor. J. Virol. 72:6104-6112.
Jia, Q., and R. Sun. 2003. Inhibition of gammaherpesvirus replication by RNA interference. J. Virol, 77:3301-3306.
Josephs, S. F. et al., 1991. HHV-6 reactivation in chronic fatigue syndrome. Lancet 337:1346-7.
Kaptein, S. J. et al., 2003. The rat cytomegalovirus R78 G protein-coupled receptor gene is required for production of infectious virus in the spleen. J. Gen. Virol. 84:2517-2530.
Kasolo, F, C>, E. Mpabalwani, and U. A. Compels. 1997. Infection with AIDS-related herpesviruses in human immunodeficiency virus-negative infants and endemic childhood Kaposi's sarcoma in Africa. J Gen Virol 78 (Pt 4):84755.
Keegan, B. and J. Noseworthy. 2002. Multiple sclerosis. Annu Rev Med 53:285-302.
Kern, E. R, et al. 2005. In vitro activity and mechanism of action of methylenecyclopropane analogs of nucleosides against herpesvirus replication. Antimicrob Agents Chemother 49:1039-45.
Kledal, T. N., M. M. Rosenkilde, and T. W. Schwartz. 1998. Selective recognition of the membrane-bound CX3C chemokine, fractalkine, by the human cytomegalovirus-encoded broad-spectrum receptor US28. FEBS Lett. 441:209-214.
Komaroff. A. L. 1988. Chronic fatigue syndromes: relationship to chronic viral infections. J Virol Methods 21:3-10.
Kong, H., Q. Baerbig, L. Duncan, N. Shepel, and M. Mayne. 2003. Human herpesvirus type 6 indirectly enhances oligodendrocyte cell death. J Neurovirol 9:539-50.
Margulies, B. J., H. Browne, and W. Gibson. 1996. Identification of the human cytomegalovirus G protein-coupled receptor homologue encoded by UL33 in infected cells and enveloped virus particles. Virology 225:111-125.
Menotti, L, et al., 1999. Trafficking to the plasma membrane of the seven-transmembrane protein encoded by human herpesvirus 6 U51 gene involves a cell-specific function present in T lymphocytes. J. Virol. 73:325-333.
Michel, D. et al., 2005. The human cytomegalovirus UL78 gene is highly conserved among clinical isolates, but is dispensable for replication in fibroblasts and a renal artery organ-culture system. J. Gen. Virol. 86:297-306.
Milne, R. S. et al., 2000. RANTES binding and down-regulation by a novel human herpesvirus-6 beta chemokine receptor. J. Immunol. 164:2396-2404.
Mirzadegan, T., G. Benko, S. Filipek, and K. Palczewski, 2003. Sequence analyses of G-protein-coupled receptors: similarities to rhodopsin. Biochemistry 42:2759-67.
Moore. G. R. 2005. Mri correlates of MS pathologic subtypes. Mult Scler 11:103-5.
Mori, Y, et al., 2002. Human herpesvirus 6 variant A but not variant B induces fusion from without in a variety of human cells through a human herpesvirus 6 entry receptor, CD46. J. Virol. 76:6750-6761.
Naesens, L., and E. De Clercq. 2001. Recent developments in herpesvirus therapy. Herpes 8:12-6.
Nakano, K. et al., 2003. Human herpesvirus 7 open reading frame U12 encodes a functional beta-chemokine receptor, J. Virol. 77:8108-8115.
Neipel, P., K. Ellinger, and B. Fleckenstein. 1991. The unique region of the human herpesvirus 6 genome is essentially collinear with the UL segment of human cytomegalovirus. J. Gen. Virol. 72:2293-2297.
Oliveira, S. A., and T. E. Shenk. 2001. Murine cytomegalovirus M78 protein, a G protein-coupled receptor homologne, is a constituent of the virion and facilitates accumulation of immediate-early viral mRNA. Proc. Natl. Acad. Sci. USA 98:3237-3242.
Opsahl, M. L., and P. G. Kennedy. 2005. Early and late HHV-6 gene transcripts in multiple sclerosis lesions and normal appealing white matter. Brain 128:516-27.
Parkhill, A. L., and J. M. Bidlack. 2002. Several delta-opioid receptor ligands display no subtype selectivity to the human delta-opioid receptor. Eur. J. Pharmacol. 451:257-264.
Pellett, P. E. et al., 1993. A strongly immunoreactive virion protein of human herpesvirus 6 variant B stain Z29: identification and characterization of the gene and mapping of a variant-specific monoclonal antibody reactive epitope. Virology 195:521-31.
Pfeiffer, B. et al., 1993. Identification and mapping of the gene encoding the glycoprotein complex gp82-gp105 of human herpesvirus 6 and mapping of the neutralizing epitope recognized by monoclonal antibodies. J Virol 67:4611-20.
Pleskoff, O. et al., 1997. Identification of a chemokine receptor encoded by human cytomegalovirus as a cofactor for HIV-1 entry. Science 276:1874-1878.
Pleskoff, O, et al., 1998. The cytomegalovirus-encoded chemokine receptor US28 can enhance cell-cell fusion mediated by different viral proteins. J. Virol. 72:6389-6397.
Pruksananonda, P. et al., 1992. Primary human herpesvirus 6 infection in young children. N Engl J Med 326:1445-50.
Qiu, X. et al., 1996. Unique fold and active site in cytomegalovirus protease. Nature 383:275-9.
Ransohoff, R. M. 2005. Immunologic correlates of MS pathologic subtypes. Mult Scler 11:101-2.
Razonable, R. et al., 2002. Selective reactivation of human herpesvirus 6 variant a occurs in critically ill immunocompetent hosts. J Infect Dis 185:110-3.
Rosenkilde, M. et al., 2001. Virally encoded 7TM receptors. Oncogene 20:1582-1593.
Salahuddin, S. et al., 1986. Isolation of a new virus, HBLV, in patients with lymphoproliferative disorders. Science 234:596-601.
Sanders, V. et al., 1996. Detection of herpesviridae in postmortem multiple sclerosis brain tissue and controls by polymerase chain reaction. J Neurovirol 2:249-58.
Schirmer, E. et al., 1991. Differentiation between two distinct classes of viruses now classified as human herpesvirus 6. Proc Natl Acad Set U S A 88:5922-6.
Sharp, P. A. 1999. RNAi and double-strand RNA. Genes Dev. 13:139-141.
Singh, N. and D. Paterson, 2000. Encephalitis caused by human herpesvirus-6 in transplant recipients: relevance of a novel neurotropic virus. Transplant 69:2474-9.
Smit, M. et al., 2003. Virally encoded G protein-coupled receptors: targets for potentially innovative anti-viral drug development. Curr. Drug Targets 4:431-441.
Snyder, S. W. et al., 1996. Initial characterization of autoprocessing and active-center mutants of CMV proteinase, J Protein Chem 15:763-74.
Soldan, S. et al., 1997. Association of human herpes virus 6 (HHV-6) with multiple sclerosis: increased IgM response to HHV-6 early antigen and detection of serum HHV-6 DNA. Nat Med 3:1394-7.
Soldan, S. S. et al., 2000. Increased lymphoproliferative response to human herpesvirus type 6A variant in multiple sclerosis patients. Ann Neurol 47:306-13.
Spear, P. G. 2004. Herpes simplex virus: receptors and ligands for cell entry. Cell Microbiol. 6:401-410.
Stevens, M. et al., 2005. Cell-dependent interference of a series of new 6-amainoquinolone derivatives with viral (HIV/CMV) transactivation. J Antimicrob Chemother 56:847-55.
Streblow, D. N. et al., 2005. Rat cytomegalovirus accelerated transplant vascular sclerosis is reduced with mutation of the chemokine-receptor R33. Am. J. Transplant. 5:436-442.
Streblow, D. et al., 1999. The human cytomegalovirus chemokine receptor US28 mediates vascular smooth muscle cell migration. Cell 99:511-520.
Takeda, K. et al., 1997. Identification of a variant B-specific neutralizing epitope on glycoprotein H of human herpesvirus-6. J Gen Virol 78 (Pt 9):2171-8.
Takeda, K. et. al., 1996. Identification of a variant A-specific neutralizing epitope on glycoprotein B (gB) of human herpesvirus-6 (HHV-6). Virology 222:176-83.
Tejada-Simon, M. V. et al., 2003. Cross-reactivity with myelin basic protein and human herpesvirus-6 in multiple sclerosis. Ann Neurol 53:189-97.
Tiley, L. et al., 1992. The VP16 transcription activation domain is functional when targeted to a promoterproximal RNA sequence. Genes Dev. 6:2077-2087.
Trkola, A. et al., 1999. The CC-chemokine RANTES increases the attachment of human immunodeficiency virus type 1 to target cells via glycosaminoglycans and also activates a signal transduction pathway that enhances viral infectivity. J. Virol. 73:6370-6379.
Vieira, J. et al., 1998. Functional analysis of the human cytomegalovirus US28 gene by insertion mutagenesis with the green fluorescent protein gene. J. Virol. 72:8158-8165.
Waldhoer, M. et al., 2003. The carboxyl terminus of human cytomegalovirus-encoded 7 transmembrane receptor US28 camouflages agonism by mediating constitutive endocytosis. J Biol Chem 278:19473-82.
Waldhoer, M. et al., 2002. Murine cytomegalovirus (CMV) M33 and human CMV US28 receptors exhibit similar constitutive signaling activities, J. Virol. 76:8161-8168.
Wang, F. et al., 1999. Human herpesvirus 6 infection inhibits specific lymphocyte proliferation responses and is related to lymphocytopenia after allogeneic stem cell transplantation. Bone Marrow Transplant 24:1201-6.
Wess, J. 1998. Molecular basis of receptor/G-protein-coupling selectivity. Pharmacol Ther 80:231-64.
Wiebusch, L., M. Truss, and C. Hagemeier. 2004. Inhibition of human cytomegalovirus replication by small interfering RNAs. J. Gen. Virol, 85:179-184.
Williams-Aziz, S. et al., 2005. Comparative activities of lipid esters of cidofovir and cyclic cidofovir against replication of herpesviruses in vitro. Antimicrob Agents Chemother 49:3724-33.
Xu, Y. et al., 2001. Definition of a divergent epitope that allows differential detection of early protein p41 from human herpesvirus 6 variants A and B. J Clin Microbiol 39:1449-55.
Yamanishi, K. et al., 1988. Identification of human herpesvirus-6 as a causal agent for exanthem subitum. Lancet 1:1065-7.
Zerr, D. M. et al., 2005. A population-based study of primary human herpesvirus 6 infection, N Engl J Med 352:768-76.
Zerr, D. M. et al., 2001. Human herpesvirus 6 reactivation and encephalitis in allogeneic bone marrow transplant recipients. Clin. Infect. Dis. 33:763-771.
Zhen, Z. et al., 2004. Transient overexpression of kappa and mu opioid receptors using recombinant adenovirus vectors. J. Neurosci. Methods 136:133-139.

1. A method of treating or preventing HHV-6 infection in a subject, comprising administering to the subject a composition comprising an inhibitor of U51.

2. The method of claim 1, wherein the inhibitor of U51 is a functional nucleic acid.

3. The method of claim 1, wherein the inhibitor of U51 is selected from the group consisting of an antisense oligonucleotide, an aptamer, and an interfering RNA (RNAi).

4. The method of claim 1, wherein the inhibitor of U51 blocks ligand binding to U51.

5. The method of claim 4, wherein the inhibitor is a derivative of a β-chemokine.

6. The method of claim 5, wherein the chemokine is RANTES, monocyte chemoattractant protein 1 (MCP-1), macrophage inflammatory protein 1α (MIP-1α) or macrophage inflammatory protein 1β (MIP-1β).

7. The method of claim 1, wherein the inhibitor inhibits U51 coupling to G-protein.

8. The method of claim 1, wherein the composition further comprises an inhibitor of HHV-6 glycoprotein gB.

9. The method of claim 1, wherein the composition further comprises an inhibitor of HHV-6 U94 gene product.

10. The method of claim 1, wherein the composition further comprises an inhibitor of viral DNA polymerase.

11. The method of claim 1, wherein the composition further comprises an inhibitor of viral primase or helicase.

12. The method of claim 1, wherein the composition further comprises an inhibitor of viral ribonucleotide reductase.

13. The method of claim 1, wherein the composition further comprises an inhibitor of HHV-6 encoded UL69 kinase.

14. The method of claim 1, wherein the composition further comprises an inhibitor of HHV-6B U83 gene product.

15. The method of claim 1, wherein the composition further comprises an inhibitor of membrane fusion.

16. The method of claim 1, wherein the composition further comprises an inhibitor of COX-2.

17. The method of claim 1, wherein the composition further comprises a HHV-6 protease blocker.

18. The method of claim 1, wherein the composition further comprises an inhibitor of viral alkaline nuclease.

19. A method of treating or preventing HHV-6 infection in a subject, comprising administering to the subject a composition comprising an inhibitor of glycoprotein gB.

20. The method of claim 19, wherein the inhibitor of HHV-6 gB is a functional nucleic acid.

21. The method of claim 19, wherein the inhibitor of HHV-6 gB is selected from the group consisting of an antisense oligonucleotide, an aptamer, and an interfering RNA (RNAi).

22. The method of claim 19, wherein the inhibitor of HHV-6 gB blocks cell fusion or ligand binding by gB.

23. The method of claim 22, wherein the inhibitor is heparin, a heparin analog or a synthetic derivative thereof.

24. The method of claim 19, wherein the composition further comprises an inhibitor of HHV-6 U51 gene product.

25. The method of claim 19, wherein the composition further comprises an inhibitor of HHV-6 U94 gene product.

26. The method of claim 19, wherein the composition further comprises an inhibitor of viral DNA polymerase.

27. The method of claim 19, wherein the composition further comprises an inhibitor of viral primase or helicase.

28. The method of claim 19, wherein the composition further comprises an inhibitor of viral ribonucleotide reductase.

29. The method of claim 19, wherein the composition further comprises an inhibitor of HHV-6 encoded UL69 kinase.

30. The method of claim 19, wherein the composition further comprises an inhibitor of HHV-6B U83 gene product.

31. The method of claim 19, wherein the composition further comprises an inhibitor of membrane fusion.

32. The method of claim 19, wherein the composition further comprises an inhibitor of COX-2.

33. The method of claim 19, wherein the composition further comprises a HHV-6 protease blocker.

34. The method of claim 19, wherein the composition further comprises an inhibitor of viral alkaline nuclease.

35. A method of inhibiting HHV-6 replication, comprising contacting HHV-6 with a composition comprising an inhibitor of U51 or glycoprotein gB.

36. A method of screening for an inhibitor of HHV-6 replication, comprising:
a) contacting a cell comprising a nucleic acid encoding HHV-6 U51 functionally linked to an expression control sequence with a candidate agent;
b) detecting U51 gene expression in the cell, wherein a decrease in U51 expression as compared to a control indicates that the candidate agent is an inhibitor of HHV-6 replication.

37. An inhibitor of HHV-6 replication identified by the method of claim 36.

38. A method of screening for an inhibitor of HHV-6 replication, comprising:
a) contacting a system comprising HHV-6 U51 and a β-chemokine with a candidate agent; and
b) detecting U51 binding to the β-chemokine, wherein a reduction in binding as compared to a control indicates that the candidate agent is an inhibitor of HHV-6 replication.

39. An isolated inhibitor of HHV-6 replication identified by the method of claim 38.

40. A composition comprising a nucleic acid, wherein the nucleic acid inhibits expression of U51.

41. The composition of claim 40, wherein the nucleic acid is an interfering RNA (RNAi).

42. An HHV-6 antibody detection kit, comprising HHV-6 polypeptides, wherein the polypeptides are selected for being highly abundant, immunodominant, and bioavailable, and labeled anti-IgG antibodies.

43. An isolated HHV-6 polypeptide, wherein the polypeptide is selected for being highly abundant, immunodominant, and bioavailable.

44. A method for detecting antibodies to HHV-6 in a sample, comprising the steps of:
a) immobilizing an HHV-6 polypeptide on a surface of a substrate;
b) administering a sample to the substrate, wherein HHV-6-specific antibodies in the sample bind the polypeptides; and
c) detecting antibody bound to the polypeptides.
